(19) United States
(12) Patent Application Publication
(10) Pub. No.: US 2010/0261213 A1
(43) Pub. Date: Oct. 14, 2010

(54) METHOD FOR LARGE SCALE PREPARATION OF THE ACTIVE DOMAIN OF HUMAN PROTEIN TYROSINE PHOSPHATASE WITHOUT FUSION PROTEIN

(76) Inventors: Seong Eon Ryu, Daejeon (KR); Dae Gwin Jeong, Daejeon (KR); Jae Hoon Kim, Jeju-do (KR); Seung Jun Kim, Gyeonggi-do (KR); Sang Jeon Chung, Daejeon (KR); Jeong Hee Son, Daejeon (KR)

Correspondence Address: KLARQUIST SPARKMAN, LLP, 121 SW SALMON STREET, SUITE 1600, PORTLAND, OR, 97204, US

(21) Appl. No.: 12/746,438

(22) PCT Filed: Aug. 4, 2008
(86) PCT No.: PCT/KR08/04524

§ 371 Date: Jun. 4, 2010

(30) Foreign Application Priority Data
Dec. 4, 2007 (KR) 10-2007-125162

(51) Int. Cl. C12Q 1/42; C12N 9/16

(52) U.S. Cl. 435/21; 435/196

(57) ABSTRACT

The present invention relates to protein tyrosine phosphatase (PTP) and a method for preparing the same, precisely, a method for expressing PTP active domain with high activity and stability without help of a fusion protein, by using computer based protein structure prediction technique. PTP prepared by the method of the present invention can be effectively used as a protein for high efficiency drug screening for the development of a novel drug, as an antigen protein for the construction of a selective antibody and as a protein for the studies of PTP structure and functions.

### TECHNICAL FIELD

The present invention relates to protein tyrosine phosphatase (PTP) and a method for preparing the same.

### BACKGROUND ART

Protein tyrosine phosphorylation-dephosphorylation plays a very important role in intracellular signal transduction system. In particular, protein tyrosine phosphorylation-dephosphorylation is involved in changes of cells such as responses to foreign stimuli, cell growth, differentiation and apoptosis, etc. Therefore, protein tyrosine kinase (PTK; Curr Pharm Des 13:2751-65, 2007; Curr Med Chem 14:2214-34, 2007) and protein tyrosine phosphatase (PTP) are important target proteins for the treatment of such diseases accompanying the change of cells as cancer, vascular disease, immune disease and nervous disease (Curr Cancer Drug Targets 6:519-532, 2006; Med Res Rev 27:553-73, 2007). Glivec, the inhibitor of abl-PTK which is one of PTKs, draws our attention as a novel drug for the treatment of chronic myeloid leukemia (Curr Opin Drug Discov Devel 7:639-48, 2004). Unlike PTK, PTP has not been explored much. But, some of PTPs are now targets of studies to treat cancer and diabetes, suggesting that PTPs have a great potential as a target protein for the treatment of such diseases.

Destruction of intracellular signal transduction system easily results in the development of a disease. So, it has been reported that PTPs have something to do with diseases and thus some of PTPs have been targets for the development of a novel drug. Humans have approximately 100 types of PTPs (Cell 117:699-711, 2004). Among these PTPs, approximately 20 PTPs have been used as a target for the development of a novel drug since their involvement in diseases was confirmed. It is thereby presumed that the remaining 80 PTPs might be involved in disease development. To develop an effective novel drug, activity of a target PTP has to be inhibited without affecting other PTPs. However, active sites of PTPs are all similar in their structures, so that a compound capable of inhibiting activity of a target PTP could inhibit activities of other PTPs. If that is the case, intracellular signal transduction network can be disturbed randomly with causing side effects with a used drug. In particular, risks of using PTPs whose intracellular functions have not been disclosed are especially great.

Therefore, it is important to develop PTP inhibitor to investigate all the activities of every PTP so as to screen a specific PTP specific compound. But, this is only possible when active protein of each PTP is identified. This active protein of each PTP is also necessary for the studies on cell functions in PTP related disease or for the development of an antibody for diagnosis of a disease. In order to use PTP for the above purposes, it is required for PTP to maintain its activity for a long time as stable as possible, and it is advantageous for PTP not to be fused with a fusion protein such as MBP and GST for the construction of an effective antibody.

Research groups have succeeded in expressing active domains sporadically and studied on the structures and functions of those active domains, which were not enough, though, and only about 20 reports have been made so far which still leave questions in activity and stability. Large scale expression of above approximately 100 PTP proteins has not been successful and the expression of 77 PTP proteins in E. coli using MBP fusion protein was successfully induced first by the present inventors (Korean Patent No. 746993). However, the use of MBP fusion protein has a problem, which is the decrease of stability after MBP elimination. So, MBP is limited in use for measuring activity level for the development of an inhibitor or for the construction of a selective antibody.

The present inventors precisely predicted N-terminal and C-terminal of PTP active domain, by taking advantage of protein structure prediction method using a computer. And the present inventors further completed this invention by confirming that 60 PTP active domains could be expressed stably without using a fusion protein only by cloning and expressing the active domains.

### DISCLOSURE

### Technical Problem

It is an object of the present invention to provide a method for preparing a recombinant PTP active domain.

It is another object of the present invention to provide a recombinant PTP active domain prepared by the method of the present invention.

It is also an object of the present invention to provide a polynucleotide encoding the above recombinant PTP active domain.

It is further an object of the present invention to provide an expression vector containing the said polynucleotide.

It is also an object of the present invention to provide a transformant transfected with the said expression vector.

It is also an object of the present invention to provide a kit for screening PTP inhibitor or activator containing the said recombinant PTP active domain.

It is also an object of the present invention to provide PTP specific antibody capable of binding specifically using the said recombinant PTP active domain.

It is also an object of the present invention to provide a method for screening PTP activity inhibitor or activator using the said recombinant PTP active domain.

It is also an object of the present invention to provide a method and kit for measuring level of PTP using the said recombinant PTP active domain.

### Technical Solution

To achieve the above objects, the present invention provides a method for preparing a recombinant PTP active domain comprising the following steps:

1) investigating homology among sub-groups of protein tyrosine phosphatase (PTP) and selecting the region exhibiting high homology;

2) examining whether the selected region of step 1) corresponds to the active domain of the standard protein whose secondary and tertiary structures have already been identified;

3) analyzing the secondary structure of the selected region of step 1) if it corresponds to the active domain and then primary determining the boundary of PTP active domain by the location not containing helix or sheet of the secondary structure;

4) secondary determining the boundary both N-terminal and C-terminal of the PTP active domain primarily determined in step 3) to be a soluble form by amino acid analysis;

5) constructing an expression vector containing a polynucleotide encoding the amino acids included in the inside of the boundary of the PTP active domain secondarily determined in step 4);

6) generating a transformant by introducing the expression vector of step 5) into a host cell; and,

7) inducing expression of the recombinant PTP active domain by culturing the transformant of step 6) and recovering thereof.

The present invention also provides a recombinant PTP active domain prepared by the method of the present invention.

The present invention further provides a polynucleotide encoding the said recombinant PTP active domain.

The present invention also provides an expression vector containing the said polynucleotide.

The present invention also provides a transformant transfected with the said expression vector.

The present invention also provides a kit for screening PTP inhibitor or activator containing the said recombinant PTP active domain.

The present invention also provides PTP specific antibody capable of binding specifically using the said recombinant PTP active domain.

The present invention also provides a method for screening PTP activity inhibitor or activator comprising the following steps:

1) treating PTP specific substrate and candidates to the PTP active domain, followed by determining activity based on optical density after measuring the optical density; and,

2) selecting candidates which reduce or increase the activity of the recombinant PTP active domain by comparing the activity of step 1) with that of the non-treated control.

The present invention also provides a method for measuring level of PTP comprising the following steps:

1) adding the PTP specific antibody of the present invention to the sample separated from a subject to conjugate PTP in samples with the antibody; and,

2) measuring a level of PTP conjugated with the antibody of step 1).

The present invention also provides a kit for measuring level of PTP which contains the PTP specific antibody of the present invention.

The present invention also provides a use of the said recombinant PTP active domain for the screening of PTP activity inhibitor or activator.

In addition, the present invention provides a use of the said PTP specific antibody for the measurement of PTP level in sample.

### ADVANTAGEOUS EFFECT

As explained hereinbefore, PTP prepared by the method of the present invention can be effectively used as a protein for high efficiency drug screening for the development of a novel drug, as an antigen protein for the construction of a selective antibody and as a protein for the studies of PTP structure and functions.

### DESCRIPTION OF DRAWINGS

The application of the preferred embodiments of the present invention is best understood with reference to the accompanying drawings, wherein:

FIG. 1 is a diagram illustrating the tertiary structure of the active domain of PTP (PTP1B: first PTP purified and identified with its characteristics).

FIG. 2 is a diagram illustrating the cleavage map of the expression vector containing the PTP active domain inserted.

FIG. 3 is a diagram illustrating the arrangement of amino acids using Clustal X program.

FIG. 4 is a diagram illustrating the prediction of the secondary structure using GOR IV SECONDARY STRUCTURE PREDICTION METHOD (//pbil.ibcp.fr/).

FIG. 5 is a diagram illustrating the prediction of hydrophilicity/hydrophobicity of the amino acid sequence using ExPASy server.

FIG. 6 is a diagram illustrating the result of SDS-PAGE with the purified protein.

FIG. 7 is a diagram illustrating the result of measurement of activity of PTP active domain (PTP1B) using DiFMUP (circle: substrate only, square: PTP1B).

FIG. 8 is a diagram illustrating the design of stable domain (T38) based on protease treatment (arrow A: location of unstable domain before protease treatment, arrow B: location of stable domain after protease treatment):

Lane 1: T38 not treated with protease; and,

Lane 2-Lane 13: T38 treated with protease with increasing the concentration.

FIG. 9 is a diagram illustrating the solubility and stability of the redesigned domain [pk7(MKP2)] (arrow: location of full length pK7):

a: solubility and stability of full length pk7; and,

(lanes 1, 3, 4 and 6: standard lanes [for pk7 location identification]; lanes 2 and 5: supernatant after cell lysis)

b: solubility and stability of redesigned pk7 domain

(lanes 1, 3, 5, 6 and 8: standard lanes; lane 2: marker; lanes 4 and 7: supernatant after cell lysis).

### BEST MODE

The terms used in this invention are described hereinafter.

“PTP active domain” indicates not full length PTP protein but a functional fragment thereof determined by the method of the present invention.

Hereinafter, the present invention is described in detail.

The present invention provides a method for preparing a recombinant PTP active domain comprising the following steps:

1) investigating homology among sub-groups of protein tyrosine phosphatase (PTP) and selecting the region exhibiting high homology;

2) examining whether the selected region of step 1) corresponds to the active domain of the standard protein whose secondary and tertiary structures have already been identified;

3) analyzing the secondary structure of the selected region of step 1) if it corresponds to the active domain and then primary determining the boundary of PTP active domain by the location not containing helix or sheet of the secondary structure;

4) secondary determining the boundary both N-terminal and C-terminal of the PTP active domain primarily determined in step 3) to be a soluble form by amino acid analysis;

5) constructing an expression vector containing a polynucleotide encoding the amino acids included in the inside of the boundary of the PTP active domain secondarily determined in step 4);

6) generating a transformant by introducing the expression vector of step 5) into a host cell; and,

7) inducing expression of the recombinant PTP active domain by culturing the transformant of step 6) and recovering thereof.

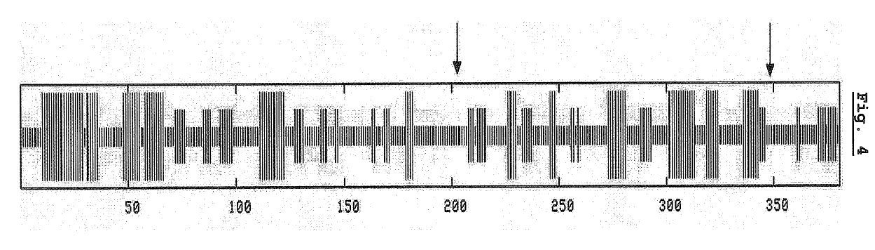

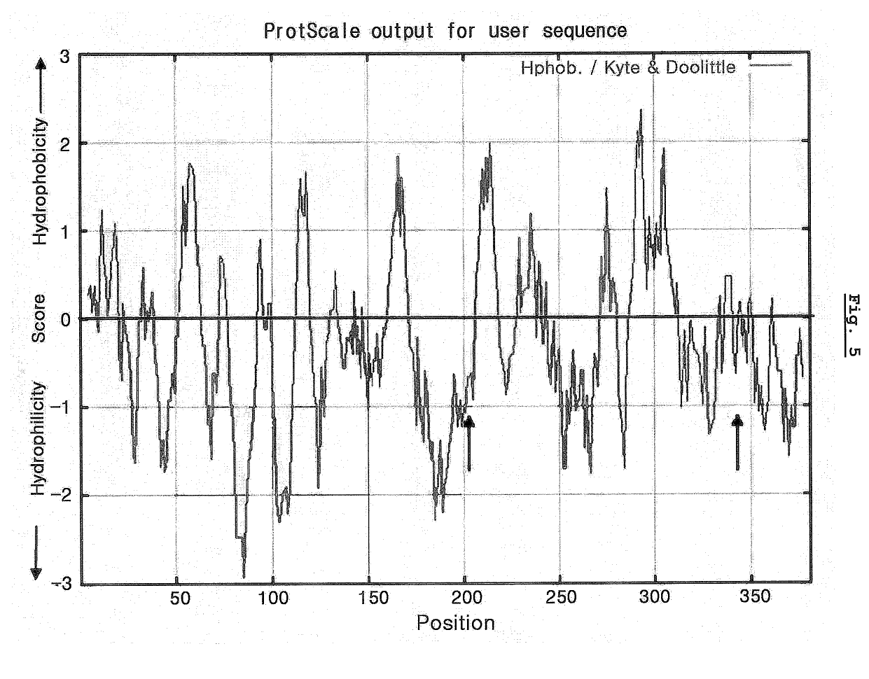

The representative tertiary structure of PTP active domain (PTP1B) is presented in FIG. 1 as the picture of ribbon. PTP has the structure in which beta-sheet in the center is surrounded with several alpha-helixes. About 100 PTPs have similar structures with this. To produce stable PTP, the present inventors compared amino acid residues of PTPs whose structures have not been disclosed with those of PTPs whose structures have already been disclosed to predict and express the presumed region of the amino acid sequence that is believed to contain a stable form of active domain (see FIG. 3 and FIG. 4).

The investigation of homology in step 1) can be performed by computer programs such as ClustalX, KALIGN (At Karolinska Institute or at EB), MAFFT (At Kyushu University, EBI or at MyHits) and Muscle (At Berkeley or at BioAssist). The sub-groups of step 1) are classified into 5 groups: receptor, non-receptor, MKP (Mitogen-Activated protein Kinase phosphatase), DUSP (Dual-specificity phosphatases) and CDCl4 (Cell division cycle 14) homologue. These 5 groups are composed of those PTPs having similar amino acid sequences and active domain structures. Therefore, based on the tertiary structures in each group of PTPs which were already identified, it was possible to predict secondary and tertiary structures of other PTPs in the same group. The identified tertiary structure in each group and PDB (Protein Data Bank) accession codes are as follows: receptor: RPTPα (1YFO) and LAR (1LAR); non-receptor: PTP1B (2HNQ) and TCPTP (1L8K); MKP: PYST1 (1MKP); DUSP: VHR (1VHR); CDC14: CDC14B (1FPZ)

The analysis of the secondary structure in step 2) can be performed by computer programs such as GOR IV SECONDARY STRUCTURE PREDICTION METHOD (//pbil.ibcp.fr/), PHDsec (//www.predictprotein.org/) and Jpred (//www.compbio.dundee.ac.uk/jpred), etc.

The boundary both N-terminal and C-terminal in step 4) is preferably determined for N-terminal and C-terminal of PTP active domain to have at least 2-3 soluble amino acids and for the start and end regions where protein folding occur to be exposed on the surface and for its secondary structure not to contain helix or sheet. The soluble amino acids herein are the amino acids having electric charge or small amino acids. The small amino acid herein is exemplified by serine or glycine. The amino acid having electric charge is exemplified by lysine, arginine, glutamine, asparagine, glutamic acid and aspartic acid.

If N-terminal and C-terminal of a recombinant protein are soluble, these terminals are easily exposed on water-soluble condition, which means these terminals can be stably expressed in an aqueous solution, and if helix or sheet structure which plays an important role in protein folding is located in the terminal of a domain, protein folding is not completed successfully and thus it is very difficult to be expressed stably in an aqueous solution.

The present inventors analyzed hydrophobic properties and secondary structure constitutions of amino acids by using ProtScale (//www.expasy.org/tools/protscale.html) of ExPASy server (Swiss Institute of Bioinformatics) (see FIG. 5).

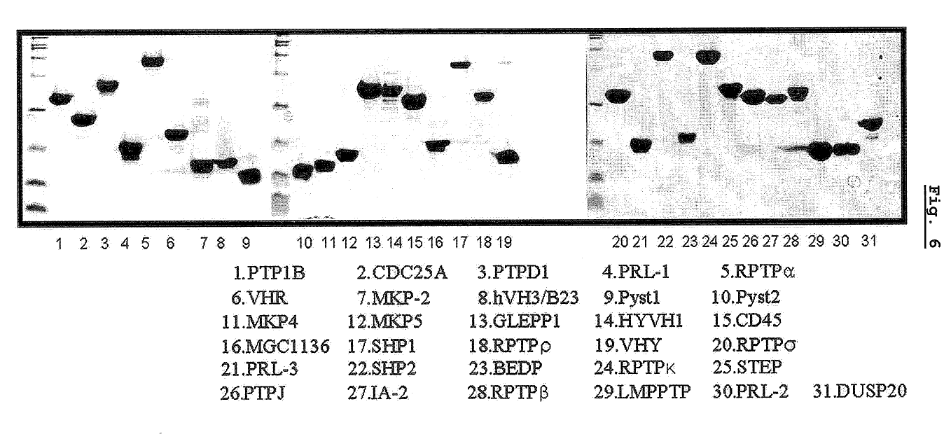

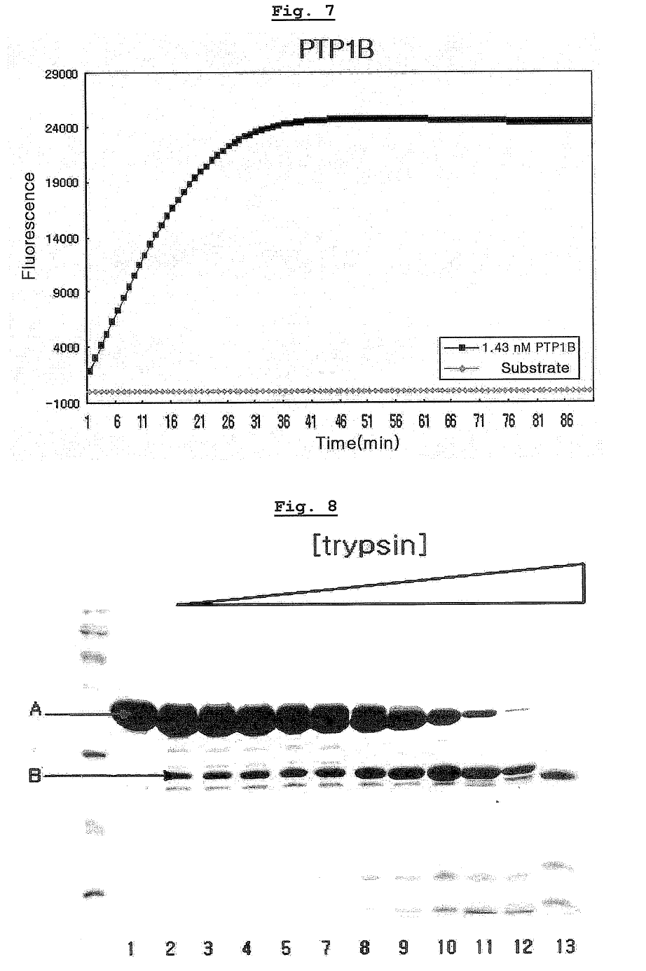

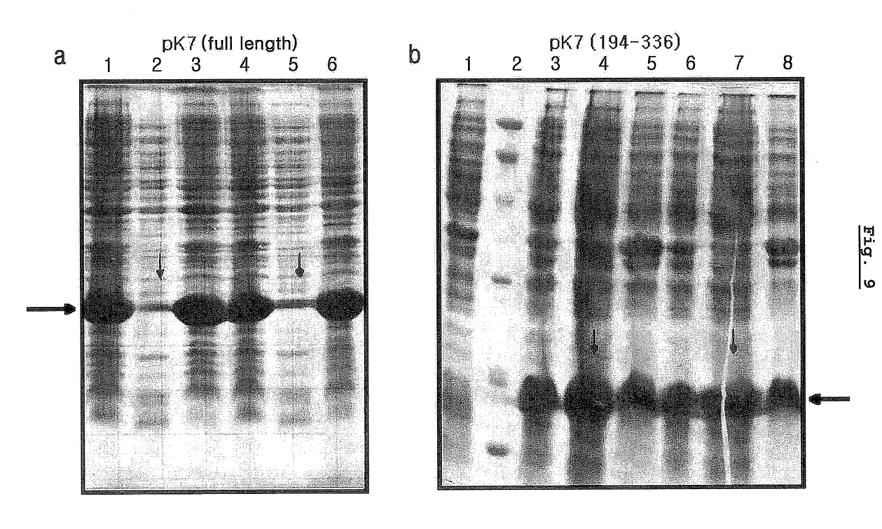

In the step of determining boundary of the active domain, an additional step of re-designing the boundary of PTP active domain may be included by treating protease, if the activity and stability of a recombinant PTP active domain are very low (see FIGS. 6 and 7). In a preferred embodiment of the present invention, PTP active domain could be re-designed to maintain activity and stability by using trypsin or chymotrypsin. The predicted boundary was hardly expressed as a stable domain at once, and after many trials of expressing different domains modified in N-terminal and C-terminal, optimum domain could be obtained. In FIG. 8, the boundary optimized for the stable expression of an active target domain is presented. So, the amino acid sequences represented by SEQ. ID. NO: 113-SEQ. ID. NO: 168 in the boundary of PTP active domain were obtained.

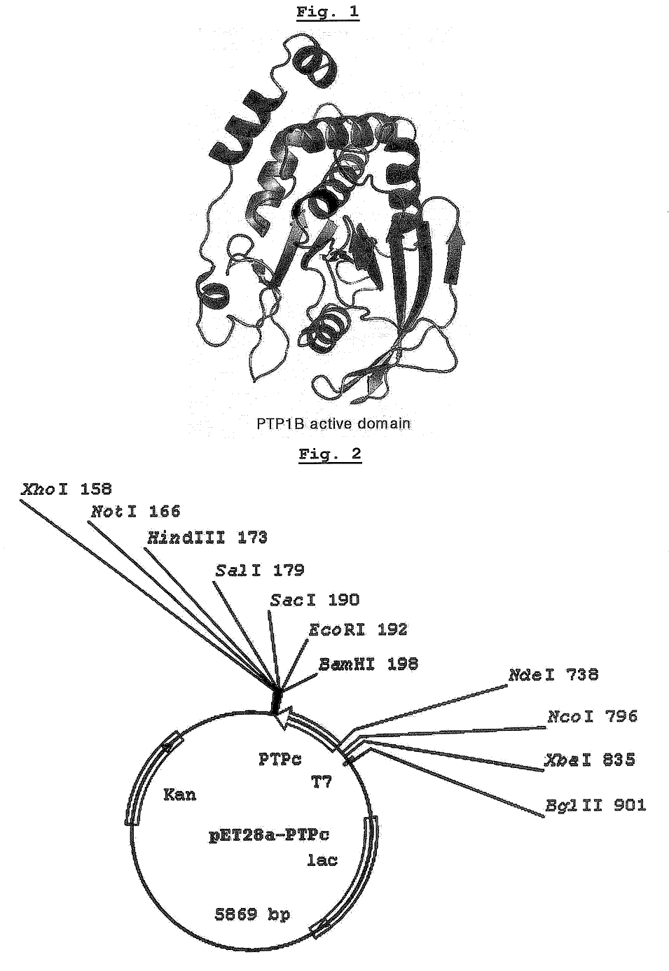

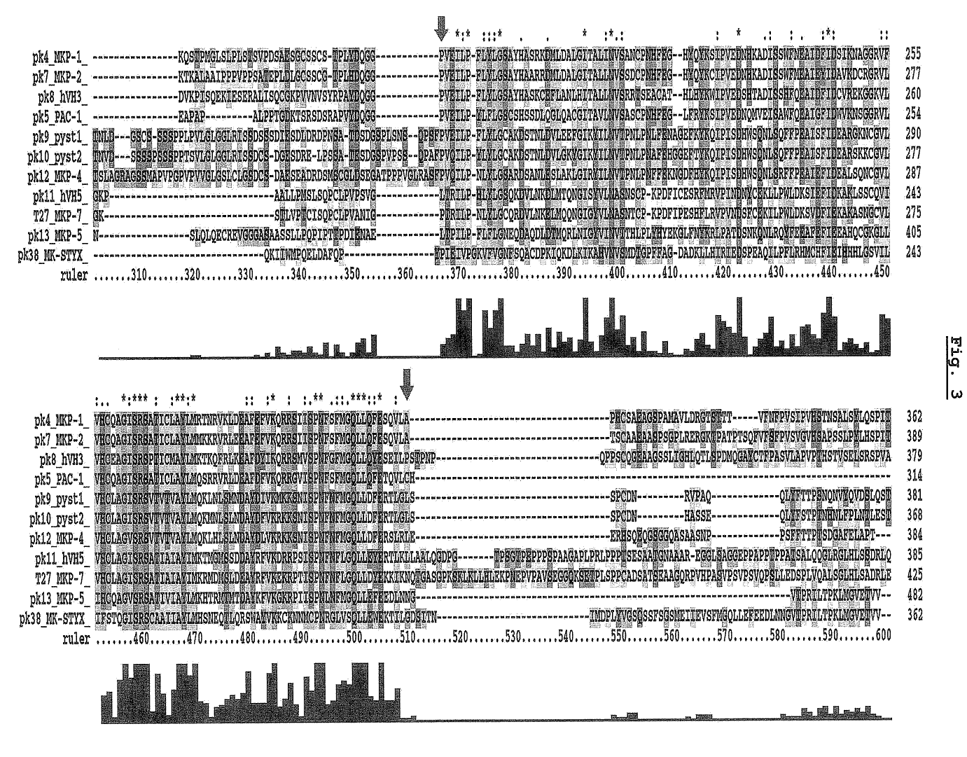

The expression vector containing a polynucleotide encoding amino acids included in the boundary of PTP active domain of step 3) is as shown in FIG. 2. The PTP active domain alone was expressed to exclude the forced link of the fusion protein with tag for separation and purification or with restriction enzyme recognition site. A region for stable PTP protein folding was determined by predicting the protein structure as described in step 1) and step 2), and expressed. Therefore, the target protein could be stably expressed as a water-soluble form by structural folding of active domain amino acid without forced linking (see FIG. 9).

In step 5), a recombinant PTP active domain was obtained under the controlled oxidation-reduction condition. In a preferred embodiment of the present invention, oxidation-reduction condition was maintained by using 5-20 mM of DTT or beta-mercaptoethanol. Approximately 30 PTP active domains were stably expressed and purified, followed by SDS-PAGE to investigate the purity of the proteins (see FIG. 9). As a result, the activity and stability remained unchanged (see FIG. 7).

The present invention also provides a recombinant PTP active domain prepared by the method of the present invention.

The PTP active domain of the present invention has high activity and stability (see FIG. 7) and retains its high stability and activity even in HTS system using hundreds of thousands of compounds, so that it can be effectively used for the studies of cell functions and disease diagnosis. The said recombinant PTP active domain comprises the amino acid sequences represented by SEQ. ID. NO: 113-SEQ. ID. NO: 168 and SEQ. ID. NO: 169-SEQ. ID. NO: 177.

The present invention further provides a polynucleotide encoding the said recombinant PTP active domain.

The present invention also provides an expression vector containing the said polynucleotide.

The vector contains the said polynucleotide in its backbone structure. The backbone vector of the present invention is preferably the vector contains restriction enzyme sites in multiple cloning sites which are generally not included in the polynucleotide encoding each polypeptide in the boundary of PTP active domains represented by SEQ. ID. NO: 113-SEQ. ID. NO: 168 and SEQ. ID. NO: 169-SEQ. ID. NO: 177, but not always limited thereto. The vector herein can be selected among various vectors capable of transfecting E. coli, such as pT7, pET/Rb, pGEX, pET28a, pET-22b(+) and pGEX. In a preferred embodiment of the present invention, polynucleotides encoding polypeptides in the boundary of PTP active domains represented by SEQ. ID. NO: 113-SEQ. ID. NO: 168 were introduced into pET28a vector (see FIG. 2) to construct expression vectors pET28a-PTP1-pET28a-PTP56 expressing the amino acids in the boundary of PTP active domains represented by SEQ. ID. NO: 113-SEQ. ID. NO: 168.

The present invention also provides a transformant transfected with the said expression vector.

The transformant herein can be effectively used for large scale preparation of PTP active domain facilitating disease diagnosis and studies of various cell functions.

The present invention also provides a kit for screening PTP inhibitor or activator containing the said recombinant PTP active domain.

The recombinant PTP active domain can be fixed on a solid carrier. The kit can additionally include a substrate for the measurement of PTP active domain activity, a reaction buffer and a reaction termination reagent, etc. The substrate herein is exemplified by DiFMUP (6,8-difluoro-4-methylumbelliferyl phosphate), OMFP (3-O-methylfluoresceinphosphate) and PTP substrate peptide labeled with fluorescent material. In a preferred embodiment of the present invention, DiFMUP was used as a substrate.

The present invention also provides PTP specific antibody capable of binding specifically using the said recombinant PTP active domain.

The antibody of the present invention can be a monoclonal antibody or polyclonal antibody. The antibody herein can be easily prepared by using the said recombinant PTP active domain of the present invention as an antigen according to the conventional antibody preparation method.

The antibody includes a polyclonal antibody, a monoclonal antibody and a fragment capable of binding to epitope.

A polyclonal antibody can be prepared as follows; one of the said recombinant PTP active domains is injected into a test animal; blood sample is taken from the animal; and then serum containing antibody is separated to isolate the antibody. Such polyclonal antibody can be purified by any methods known to those in the art and can be produced from host animals which are exemplified by goat, rabbit, sheep, monkey, horse, pig, cow, dog, etc.

A monoclonal antibody can be prepared by any method that facilitates the production of antibody molecules via culturing the continuous cell line. The method is exemplified by hybridoma technique, human-B-cell hybridoma technique, and EBV-hybridoma technique, but not always limited thereto (Kohler G et al., Nature 256:495-497, 1975; Kozbor D et al., J Immunol Methods 81:31-42, 1985; Cote R J et al., Proc Natl Acad Sci 80:2026-2030, 1983; [symbol] Cole S P et al., Mol Cell Biol 62:109-120, 1984).

An antibody fragment containing a specific binding site for one of the said recombinant PTP active domains can be prepared. For example, F(ab′)2 fragment can be prepared by fractionation of an antibody molecule by using pepsin and Fab fragment can be prepared by reducing disulfide bridge of F(ab′)2 fragment, but not always limited thereto. Alternatively it is also possible to identify a monoclonal Fab fragment having desired specificity by constructing Fab expression library (Huse W D et al., Science 254: 1275-1281, 1989).

The present invention also provides a method for screening PTP activity inhibitor or activator comprising the following steps:

1) treating PTP specific substrate and candidates to the PTP active domain, followed by determining activity based on optical density after measuring the optical density; and,

2) selecting candidates which reduce or increase the activity of the recombinant PTP active domain by comparing the activity of step 1) with that of the non-treated control.

The candidate of step 1) can be selected from the group consisting of natural compounds, synthetic compounds, RNA, DNA, polypeptides, enzymes, proteins, ligands, antibodies, antigens, metabolites of bacteria and fungi and bioactive molecules, but not always limited thereto.

The present invention also provides a method for measuring level of PTP comprising the following steps:

1) adding the PTP specific antibody of the present invention to the sample separated from a subject to conjugate PTP in samples with the antibody; and,

2) measuring a level of PTP conjugated with the antibody of step 1).

In step 1), the sample can be selected from the group consisting of blood, tissues and exudates. In step 2), the measurement is performed by a method selected from the group consisting of Western blotting, ELISA (enzyme-linked immunosorbent assay), colorimetric method, electrochemical method, fluorimetric method, luminometry, particle counting method, visual assessment and scintillation counting method.

The present invention also provides a kit for measuring level of PTP which contains the PTP specific antibody of the present invention.

The antibody herein can be fixed on a solid substrate for the convenience in washing, separation of a complex and the following steps. The solid substrate is exemplified by synthetic resin, nitrocellulose, glass plate, metal plate, microsphere and microbead, etc. The synthetic resin herein is exemplified by polyester, polyvinyl chloride, polystyrene, polypropylene, PVDF and nylon.

To mix the sample separated from a subject with the PTP specific antibody of the present invention, the sample can be diluted before the mixing. The sample can be pre-treated in order to increase PTP sensitivity by anion exchange chromatography, affinity chromatography, size exclusion chromatography, liquid chromatography, sequential extraction or gel electrophoresis, etc, but not always limited thereto.

The kit of the present invention can contain a ligand suitable for conjugating PTP specific antibody. The ligand herein is preferably secondary antibody which is specific for protein A or antibody for detection. The PTP specific antibody and ligand of the present invention can be conjugates labeled with coloring enzyme, fluorescein, isotope or colloid as probe for detection. The PTP specific antibody is preferably treated by biotinylation or with digoxigenin to be conjugated with the ligand, but the treatment method is not limited thereto. The ligand is preferably treated with streptavidin or avidin to be conjugated with PTP specific antibody, but not always limited thereto.

The kit for measuring the level of PTP active domain of the present invention is designed to screen the amount of PTP specific antibody and PTP specific antibody in the PTP complex in the sample. The kit is also capable of measuring the level of PTP by screening the ligand treated with the said antibody and PTP complex in the sample. The measurement or detection of PTP specific antibody and ligand is performed by fluorescence, iluminescence, chemiluminescence, optical density, reflection or transmission.

To screen the PTP specific antibody or ligand, high throughout screening (HTS) system is preferably used. At this time, fluorescence assay detecting fluorescence with fluorescent material labeling as probe for detection; radio assay detecting radioactive rays with isotope labeling as the probe; SPR (surface plasmon resonance) method measuring real time changes of Plasmon resonance on the surface without labeling; or SPRI (surface plasmon resonance imaging) method is used, but not always limited thereto.

For the fluorescence assay, an antibody for detection is labeled with a fluorescent material and then spotted, and signal is detected by fluorescent scanner program. The fluorescent material herein is preferably selected from the group consisting of Cy3, Cy5, poly L-lysine-fluorescein isothiocyanate (FITC), rhodamine-B-isothiocyanate (RITC) and rhodamine, but not always limited thereto. The SPR system facilitates real-time analysis of level of an antibody conjugation without fluorescent material labeling. But, it cannot facilitate simultaneous analysis of different samples. The SPRI can be used for simultaneous analysis of different samples but sensitivity is low.

The present invention also provides a use of the said recombinant PTP active domain for the screening of PTP activity inhibitor or activator.

In addition, the present invention provides a use of the said PTP specific antibody for the measurement of PTP level in sample.

The sample is tissues or body fluids including blood, urine and tear.

### MODE FOR INVENTION

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Example 1

### Determination of Boundary of N-Terminal and C-Terminal of PTP Active Domain

### <1-1> Comparison of PTP Amino Acid Sequences and Prediction of Structure

PTP active domains are classified into 5 groups: receptor, non-receptor, MKP (map kinase phosphatase), DUSP (dual-specificity phosphatases) and CDC14 (cell division cycle 14) homologue, followed by comparison of their amino acid sequences. The structures of these 5 groups were predicted based on the homology of their amino acid sequences, which were used for dividing PTP subgroups (Alonso et al., Cell 117:699-711, 2004). Based on the tertiary structures already identified [receptor: RPTPa (1YFO); non-receptor: PTP1B (2HNQ) and TCPTP (1L8K); MKP: PYST1 (1MKP); DUSP: VHR (1VHR); CDC14: CDC14B (1FPZ)], amino acid sequences of each group were arranged by using Clustal X program (FIG. 3). Particularly, 11 MKPs were analyzed by Clustal X program and high homology region (red arrow in FIG. 3) was selected, followed by determining active domain using the secondary and tertiary structures of the standard protein MKP3(pk9).

At the same tine, the secondary structure was predicted by using GOR IV SECONDARY STRUCTURE PREDICTION METHOD (//pbil.ibcp.fr/). FIG. 4 illustrates the result of secondary structure prediction of the full length standard protein MKP3(pk). Blue rod indicates alpha-helix, red rod indicates beta-sheet and purple rod indicates loop or flexible region, and blue arrow indicates the boundary of real tertiary structure. From the above results, the boundary of PTP active domain was outlined.

### <1-2> Determination of Boundary of N-Terminal and C-Terminal of PTP Active Domain

For the stable expression in aqueous solution, it is preferred for N-terminal and C-terminal of a protein to be composed of water-soluble amino acids. So, hydrophobicity and secondary structure of the amino acid were analyzed by using ProtScale (//www.expasy.org/tools/protscale.html) of ExPASy server (Swiss Institute of Bioinformatics). For example, based on the prediction of hydrophilic/hydrophobic region of the amino acid sequence of MKP3(pk9) by ExPASy server, the boundary of hydrophilicity (FIG. 5, red arrow) was selected as a domain (FIG. 5). The selected domain has very low chance of having helix or sheet structure in N-terminal and C-terminal, suggesting high chance of avoiding structural folding. If a region that contains structural folding is selected for the terminal of protein, the folding of the expressed protein therein would be unsuccessful and thus unstable in aqueous solution. Therefore, the starting region and end region of protein folding has to be exposed. To be exposed at least 2-3 amino acids of N-terminal and C-terminal on the surface, it is advantages for the N-terminal and C-terminal to have soluble amino acids and not to have helix or sheet structure in their secondary structures. It is better for the N-terminal or C-terminal to have small amino acids such as serine or glycine, amino acids having electric charge and soluble amino acids, which favors stable domain formation.

Based on the above prediction, 1-52 amino acid sequences with modified boundary to increase solubility were obtained.

### <1-3> Re-Design of Domain Boundary for the Improvement of Solubility and Stability

### <1-3-1> Confirmation of Solubility and Stability

After cloning the PTP active domain determined in Example <1-2>, it was expressed in E. coli and purified therefrom. After storing for a while, a proper amount of protein solution was ultra-centrifuged to separate supernatant and precipitate. SDS-PAGE was performed with the precipitate by the same manner as described in Example 3 to investigate whether the precipitate contained the target protein, leading to the examination of solubility.

### <1-3-2> Stable Active Domain Boundary

Based on the result of Example <1-3-1>, 20 μg of PTP active domain having low solubility and stability was serially diluted from 1:1 to 1:1,000, followed by reaction with trypsin (Sigma, USA) or chymotrypsin (Sigma, USA) at 37° C. for 30 minutes. SDS-PAGE was performed by the same manner as described in Example 3 to confirm digestion.

As a result, it was confirmed that stability of T38 was maintained even with the increase of protease concentration (FIG. 8).

### <1-3-3> Re-Design of Domain Boundary

The stable PTP active domain obtained in Example <1-3-2> was modified and reformed by N-terminal sequencing and mass spectrometry.

The protein band cut by protease obtained in Example <1-3-2> was transferred to PVDF membrane. The band was cut and treated with a reagent recognizing and digesting N-terminal, followed by HPLC stepwise to arrange amino acids. Mass spectrometry was performed with the band to calculate the mass exactly and select stable domains. The re-designed domains were tested for activity and stability by the same manner as described in Example 4.

As a result, as shown in FIG. 9, the re-designed domain pk7(MKP2) was confirmed. Particularly, as shown in FIG. 9a, solubility and stability of the full length pk7 were low. But, as shown in FIG. 9b, the re-designed pk7 demonstrated high solubility and stability. That is, the first expression with low solubility improved to the stable and increased expression of PTP active domain. The re-designed stable domains are shown in Table 1.

TABLE 1
Re-designed stable domains

| | Unstable | Stable | SEQ. |
|---|---|---|---|
| PTP name | domain | domain | ID. NO |
| p18 | 299-457 | 306-450 | 158 |
| pk14 | 1-210 | 27-210 | 145 |
| pk17 | 35-211 | 35-211 | 155 |
| pk32 | 1-360 | 63-360 | 130 |
| T20 | 840-1400 | 890-1180 | 125 |
| T23 | 1042-1305 | 1024-1335 | 117 |
| T38 | 636-979 | 709-979 | 120 |
| Eya2 | 339-514 | 244-514 | 168 |
| pK7 | 1-394 | 174-338 | 136 |

### Example 2

### Large Scale Expression and Purification of PTP Active Domain

### <2-1> Cloning of PTP Active Domain

Expression vectors capable of expressing 1-56 PTP active domains determined in Example 1 without help of a fusion protein were constructed.

The multiple cloning sites of PET28a (Novagen, USA) contains those restriction enzyme sites not included in DNA sequences of PTP active domains (SEQ. ID. NO: 113-SEQ. ID. NO: 168) most, so that it was used as a backbone vector of the present invention. As shown in Table 2, to amplify DNA sequences of PTP active domains 1-56 represented by SEQ. ID. NO: 113-SEQ. ID. NO: 168, PCR was performed with primers represented by SEQ. ID. NO: 1-SEQ. ID. NO: 112 using cDNA libraries of brain, muscle and testis purchased from Clontech as template DNAs as follows; at 95° C. for 5 minutes, at 95° C. for 1 minute, at 55-60° C. for 1 minute, at 72° C. for 90 seconds (30 cycles) and at 72° C. for 10 minutes. The amplified PCR products were digested with NdeI, EcoRI or BamHI, which were inserted into pET28a vector (Novagen, USA) and then named respectively pET28a-PTP 1-56 (FIG. 2).

TABLE 2

Nucleotide sequences of PTP active
domain 1 - 56 and primer sets

| | | | | |
|---|---|---|---|---|
| | | Amino acid | | |
| | | location | | |
| | | (SEQ. ID. NO) | | SEQ. |
| | | DNA | Forward primer | ID |
| No. | Name | location | Reverse primer | NO |

| | | | | |
|---|---|---|---|---|
| 1 | T4 | 225- | CGCGACGCTAGCATGGCAGACGACAATAAGCTCTTC | 1 |
| | | 793 (113) | | |
| | | 673-2379 | GCTGCGAAGCTTTACTTGAAGTTGGCATAATCTGA | 2 |
| 2 | T7 | 1684- | GGCACCCATATGCTAGTGGCTGTTGTTGCCTTATTG | 3 |
| | | 1967 (114) | | |
| | | 5050-5901 | GCGGGATCCTCAATGCCTTGAATAGACTGGATC | 4 |
| 3 | T48 | 1316- | GCCCCACATATGCGAGACCACCCACCCATCCCC | 5 |
| | | 1897 (115) | | |
| | | 3946-5691 | GGAAGATCTCTACGTTGCATAGTGGTCAAAGCTGCC | 6 |
| 4 | T8 | 821- | GCGCCATATGGCAGACAAGTACCAGCAACTCTCCCTG | 7 |
| | | 1089 (116) | | |
| | | 2461-3267 | GCGCGGATCCCTCGGCTGGGGCCTGGGCTGACTGTTG | 8 |
| 5 | T23 | 1024- | CCGTTACATATGGTGGAGAATTTTGAGGCCTACTTC | 9 |
| | | 1335 (117) | | |
| | | 3070-4005 | CCCGAATTCTTAGGCGATGTAACCATTGGTCTTTC | 10 |
| 6 | T39 | 879- | CACATTGCTAGCATGAAGACATCAGACAGCTATGGG | 11 |
| | | 1440 (118) | | |
| | | 2635-4320 | CGGCTCAAGCTTCTAAGATGATTCCAGGTACTCCAA | 12 |
| 7 | T5 | 848- | GCCCACCATATGGCCAGCGATACCAGCAGCCTG | 13 |
| | | 1452 (119) | | |
| | | 2542-4356 | GCGAGATCTTCAGCCAGAATTCAAGTATTCCAG | 14 |
| 8 | T38 | 709- | GACCGGCATATGCTTGCCAAGGAGTGGCAGGCCCTC | 15 |
| | | 979 (120) | | |
| | | 2125-2935 | CCGGGATCCTCACTGGGGCAGGGCCTTGAGGAT | 16 |
| 9 | T12 | 674- | CGCCAGCATATGGCCACGCGGCCACCAGACCGA | 17 |
| | | 1015 (121) | | |
| | | 2020-3045 | GCGGGATCCTCACTGGGGAAGGGCCTTGAGGAT | 18 |
| 10 | T15 | 851- | GAGCATGCTAGCATGGCTAGGGAGTGTGGAGCTGGT | 19 |
| | | 1216 (122) | | |
| | | 2551-3648 | GCGGGATCCCTAGGACTTGCTAACATTCTCGTATAT | 20 |
| 11 | T10 | 327- | CCTTTCCATATGAAGCCCATAGGACTTCAAGAGAGAAG | 21 |
| | | 650 (123) | | |
| | | 979-1950 | GACAGTAAGCTTTCAAAGTCTGCTCTCATACAGGCACA | 22 |
| 12 | T22 | 1367- | CGCGAACATATGCTTAGCCACCCGCCAATTCCC | 23 |
| | | 1650 (124) | | |
| | | 4099-4950 | GGCGGATCCTCAGCCCACGGCCTCCAGCAGGGCCTC | 24 |
| 13 | T20 | 890- | TTCGCTAGCGCCATCCGGGTGGCTGACTTG | 25 |
| | | 1180 (125) | | |
| | | 2668-3540 | GCGGGATCCCTAAAAGGAGCTTAAATATTCCAGTGCCA | 26 |
| 14 | PTP | 1-299 (126) | ATGGAGATGGAAAAGGAGTTCGAGCAGATC | 27 |
| | 1B | 1-897 | GTCAACATGTGCGTGGCTACGGTCCTCACG | 28 |
| 15 | T25 | 1-387 (127) | GCTCCCGCTAGCATGCCCACCATCGAGCGGGAG | 29 |
| | | 1-1161 | CGCGGATCCTTAGGTGTCTGTCAATCTTGGCCT | 30 |
| 16 | T41 | 157- | TCAGAGCATATGGAGGAGAAGATCGAGGATGAC | 31 |
| | | 537 (128) | | |
| | | 469-1611 | GTGGACGCTAGCATGAAATATTTGGGCAGTCCCATT | 32 |
| 17 | T18 | 1-595(129) | GCCCCCCATATGGTGAGGTGGTTTCACCGAGAC | 33 |
| | | 1-1785 | CCGGAATTCTCACTTCCTCTTGAGGGAACCCTTG | 34 |
| 18 | pk32 | 63-360 (130) | GAACCCCATATGTCTGTGAACACACCCCGGGAGGTC | 35 |
| | | 187-1080 | CGGGATCCTCAGGGGCTGGGTTCCTCAGGCAG | 36 |
| 19 | pk28 | 1-526 (131) | CCGCGGCATATGGAACATCACGGGCAATTAAAA | 37 |
| | | 1-1578 | CGGGATCCTCACCTGCAGTGCACCACGACCGG | 38 |
| 20 | T32 | 2095- | GCAGTACATATGAATGGGAAGTTATCAGAAGAG | 39 |
| | | 2490 (132) | | |
| | | 6283-7468 | GGCGGATCCTCACTTCAGAAGCTGAGGCTGCTGTTTTT | 40 |
| 21 | T40 | 866- | GAGCAGCATATGGCAGGCCTGGAGGCACAGAAG | 41 |
| | | 1187 (133) | | |
| | | 2596-3561 | CGCGGATCCTTAAATGAGTCTGGAGTTTTGGAG | 42 |
| 22 | T2 | 839- | CTAGGGCATATGAAAAAGACTCGAGTAGATGCA | 43 |
| | | 1174 (134) | | |
| | | 2515-3522 | CGCGGATCCTTAGATGAGCCTGGAGCTTTTCAG | 44 |
| 23 | pk4 | 173- | AGGCCGCATATGGTCATGGAAGTGGGCACCCTG | 45 |
| | | 323 (135) | | |
| | | 517-969 | GGCGGATCCTCAGCTCCCAGCCTCTGCCGAACAG | 46 |
| 24 | pk7 | 174- | GTTCATATGAGTGCCACAGAGCCCTTGGAC | 47 |
| | | 338 (136) | | |
| | | 520-1012 | GCGGGATCCTCAGGACGTGGCCAGCACCTGGGACTC | 48 |
| 25 | pk8 | 178- | GCGGACCATATGGGCCCAGTTGAAATCCTTCCCTTC | 49 |
| | | 321 (137) | | |
| | | 532-962 | GCGAGATCTTCACGTGGAGGGCAGGATCTCAGATTCG | 50 |
| 26 | pk9 | 205- | GGCAGCCATATGTCCTTCCCAGTGGAGATCTTGCCC | 51 |
| | | 348 (138) | | |
| | | 613-1044 | CGCGGATCCTCAGCTGAGTCCCAGCGTCCTCTCGAA | 52 |
| 27 | pk10 | 192- | GCTGGCCATATGTTGCGCCGCCTGCGCAAGGGC | 53 |
| | | 338 (139) | | |
| | | 574-1014 | CGGGATCCTCACGTGGACTCCAGCGTATTGAG | 54 |
| 28 | T33 | 160- | TGCCCCCATATGGCTGGGGACCGGCTCCCGAGG | 55 |
| | | 312 (140) | | |
| | | 478-934 | GCGGGATCCTCATGAGGGGGTGCCCGGGTCGCCCTG | 56 |
| 29 | pk12 | 201- | CGATCGCATATGGAGGGTCTGGGCCGCTCGTG | 57 |
| | | 351 (141) | | |
| | | 601-1053 | CGGGATCCCTAGGTGGGGGCCAGCTCGAAGG | 58 |
| 30 | pk13 | 320 | CTGGACCATATGCAGCGGCTGAACATCGGCTAC | 59 |
| | | 467 (142) | | |
| | | 958-1401 | CGGGATCCTCACACAACCGTCTCCACTCCCATC | 60 |
| 31 | T27 | 192- | GTTGCCCATATGGGGCCAACCCGAATTCTTC | 61 |
| | | 339 (143) | | |
| | | 574-1017 | GGATCCTTATGATGCTCCAGTCTGGTTC | 62 |
| 32 | pk6 | 1-185 (144) | GCCGCCCATATGTCGGGCTCGTTCGAGCTCTCG | 63 |
| | | 1-555 | CGGGATCCCTAGGGTTTCAACTTCCCCTCC | 64 |
| 33 | pk14 | 27-210 (145) | GCCAAGCATATGGGCGGAAACCACATCCCCGAAAGG | 65 |
| | | 79-628 | GCGGGATCCTCAGGAATTCCAATTCTTTCTGATAGG | 66 |
| 34 | pk15 | 21-340 (146) | AGCGCCCATATGGTCAGCTGTGCCGGGCAGATGCTG | 67 |
| | | 61-1020 | CGGGATCCTCATATTTTTCCTGTTTGTGATCC | 68 |
| 35 | pk33 | 1-188 (147) | GGCTGGCATATGGCTGAGACCTCTCTCCCAGAG | 69 |
| | | 1-564 | CGGGATCCTCAGCTCTGGCCGGCACCCCGC | 70 |
| 36 | p44 | 1-198 (148) | TCCCACCATATGGACTCACTGCAGAAGCAGGAC | 71 |
| | | 1-601 | GCCAAGGGTCAGGGATCCTGGCTG | 72 |
| 37 | p21 | 1-157 (149) | CCCGGGCATATGGGCAATGGCATGACCAAGGTAC | 73 |
| | | 1-371 | GCGGGATCCTCACTTGCCGCCCTTGCGGGACAG | 74 |
| 38 | pk35 | 1-188 (150) | GCGGGATCCTCACTTGCCGCCCTTGCGGGACAG | 75 |
| | | 1-564 | CGGGATCCTCACAGTGGAATCATCAAACGGAC | 76 |
| 39 | NE1 | 1-217 (151) | CCAGGGGCTAGCCGCTAACTGGAAAGAAAA | 77 |
| | | 1-651 | GTCGGATCCTTAGCTTTCTTTGCCCTCTTG | 78 |
| 40 | p19 | 1-190 (152) | ATGACAGCATCCGCGTCCTCCTTTTC | 79 |
| | | 1-570 | TTACATTGATATCATCATACGTAG | 80 |
| 41 | pk18 | 1-184 (153) | GCAGCCCATATGGGGAATGGGATGAACAAGATC | 81 |
| | | 1-552 | CGGGATCCTTACAGTCTTCTGAGAAAGGCCCAG | 82 |
| 42 | p12 | 31-211 (154) | GGGAAGCATATGGGTCGGGCGCACCGGGACTGG | 83 |
| | | 91-603 | GGCACCAAGCTTTCAGAACTCTTTAAGAACATCCAGCT | 84 |
| 43 | pk17 | 35-211 (155) | CTGGAGCATATGCCAACCGTTCAACATCCTTTCC | 85 |
| | | 103-633 | GCGGGATCCTCATGCTTCCAGACCCTGCCGCAGC | 86 |
| 44 | p16 | 1-150 (156) | GCGGCGGCTAGCATGGGCGTGCAGCCCCCCAACTTC | 87 |
| | | 1-350 | CGCGCCTCGAGTTTCGTTCGCTGGTAGAACTGGAA | 88 |
| 45 | T16 | 1-210 (157) | GGCGGCGCTAGCATGGCTCACAACAAGATCCCGCCG | 89 |
| | | 1-630 | TGAGGATCCTTATGATTCCTTCTTTCCATCCTCATC | 90 |
| 46 | p18 | 306- | CCGGGACATATGGACAAGCCCTCCCTTATCTTC | 91 |
| | | 450 (158) | | |
| | | 916-1350 | GCGGGATCCTCAGCTTGCATCCAAGATGCCTTC | 92 |
| 47 | NE3 | 306- | CTTGGTCATATGGATAGCCCTACACAGATATTTG | 93 |
| | | 350 (159) | | |
| | | 916-1350 | GCGGGATCCTCACCTTGCCAGCAAGATCCCCTG | 94 |
| 48 | pk3 | 4-163 (160) | GCGGCTCATATGAACCGCCCAGCTCCTGTGGAA | 95 |
| | | 10-489 | GCGGGATCCTCAGGAATCTTTGAAACGCAGCCGCAT | 96 |
| 49 | p49 | 14-167 (161) | CGCCGAGCTAGCATGCGTTTTCTGATAACTCACAAC | 97 |
| | | 40-501 | CGGGATCCCTACTGAACACAGCAATGCCCATTG | 98 |
| 50 | p26 | 4-161 (162) | GCGACCCATATGGCCCCGGTGGAGGTGAGCTACA | 99 |
| | | 10-483 | CGCGGATCCTCAGGTCTTGTGCGTGTGTGGGTCTTTG | 100 |
| 51 | T29 | 37-391 (163) | GGCGGCCATATGTCGTCGACCTCGCCGGGTGTGAAG | 101 |
| | | 109-1173 | GCCGGATCCTTATTTGGAGAAGGCTGCTCTGTGTTGTC | 102 |
| 52 | T46 | 1-157 (164) | ATGGCGGAACAGGCTACCAAGTCCGTG | 103 |
| | | 1-371 | TCAGTGGGCCTTCTCCAAGAACGCTCTGC | 104 |
| 53 | pk1 | 336- | GCTCTAGACTTATAGGAGACTTCTCCAAGGG | 105 |
| | | 523 (165) | | |
| | | 1006-1569 | GCCCTAGGTCAGAGCTTCTTCAGACGACTGTAC | 106 |
| 54 | T47 | 378- | GACCACCATATGCTGATTGGAGATTACTCTAAGGCC | 107 |
| | | 566(166) | | |
| | | 1132-1701 | CCGGGATCCTCACTGGTCCTGCAGCCGGCTACA | 108 |
| 55 | T45 | 207- | GATTCTGCTAGCGGGCACCTGATTGGTGATTTTTCC | 109 |
| | | 400 (167) | | |
| | | 619-1200 | CCGGGATCCTCATGGGCTCATGTCCTTCACCAG | 110 |
| 56 | Eya2 | 244- | GACAATCATATGGAGCGTGTGTTCGTGTGGGAC | 111 |
| | | 514 (168) | | |
| | | 730-1542 | GAATTCTTATAAATACTCCAGCTCCAGGGCGTG | 112 |

<2-2> Conditions for Large Scale Expression with Maintaining Activity and Stability

E. coli was transfected respectively with the 56 vectors constructed in Example <2-1> according to the method of Hanahan (Hanahan D, DNA Cloning vol. 1 109-135, IRS press 1985).

Particularly, E. coli BL21-DE3-RIL treated with CaCl₂ was transfected with vectors constructed in Example <2-1> by heat-shock method. Then, the cells were cultured in medium containing kanamycin (Sigma, USA). Colonies having kanamycin resistance were selected. These colonies were cultured in LB medium for overnight and then some of the seed culture solution was inoculated in LB medium containing 30 μg/ml of kanamycin, followed by culture until stationary phase. The culture solution was diluted at the ratio of 1:100 and inoculated in fresh LB medium (400 ml/flask). Temperature was lowered slowly from 37° C. to 17° C. during 2-3 hour culture. Then, culture was continued at 17° C. at 200 rpm. When OD₆₀₀ of the culture solution reached 0.5, IPTG was added at the lowest concentration (0.05-0.1 mM), followed by further culture for 20 or 16-18 hours to induce expression of PTP active domain.

<2-3> Conditions for Purification and Storage with Maintaining Activity and Stability

E. coli cultured in Example <2-2> was centrifuged at 4° C. at 6,000 rpm for 5 minutes. The cell precipitate was recovered, which was resuspended in 5 ml of cell lysis buffer (10 mM Tris-HCl buffer, pH 7.5, 10 mM EDTA). The cells were lysed using ultrasonicator at 4° C. Centrifugation was performed at 4° C. at 13,000 rpm for 10 minutes to separate supernatant and insoluble aggregate. Protein was eluted from the supernatant by linear density gradient using Ni-NTA resin (Qiagen, USA) at 4° C. for about 3 hours from low concentration buffer [20 mM Tris-HCl buffer, pH 7.5, 0.2 M NaCl, 1.0 mM PMSF, 4 mM β-mercaptoethanol (Sigma, USA)] to high concentration buffer [0.5 M imidazole (Sigma, USA) was added to the low concentration buffer]. The histidine tag of N-terminal of the eluted protein was eliminated by treating thrombin (protease) (Sigma, USA) by 1 unit/100 μg protein. The protein was purified by ion exchange chromatography (GE Healthcare, USA) and gel filtration chromatography (GE Healthcare, USA). During the purification of PTP active domain, 10 mM β-mercaptoethanol (Sigma, USA) or DTT (Promega, USA) was added to the buffer and pH of the buffer was regulated to 6.5-8.0. The purified PTP active domain was stored at 4° C. with the addition of 10% glycerol in protein solution [10% glycerol solution prepared by adding 100-250 mM NaCl, 10 mM reducing agent (β-mercaptoethanol or DTT) and 0.5˜2 μg/ml protease inhibitor (Sigma, USA) to pH 7.5-8.0 Tris buffer].

### Example 3

### SDS-PAGE with PTP Active Domain

The results (size and purity of protein) of purification of PTP active domain obtained in Example 2 were confirmed by SDS-PAGE.

The concentration of PTP active domain obtained by the method of Example 2 was measured by using Bio-Rad protein assay kit. The protein was mixed with 5×SDS (0.156 M Tris-HCl, pH 6.8, 2.5% SDS, 37.5% glycerol, 37.5 mM DTT) at the ratio of 1:4, followed by boiling at 100° C. for 10 minutes. 1-2 μg of the boiled sample was loaded in each well of 10% SDS-PAGE gel, followed by developing at 125 V for 2 hours. After Coomassie staining, destaining was performed and expression of each recombinant protein was examined.

As a result, as shown in FIG. 6, based on the size measured, the protein was confirmed to be PTP active domain having at least 95% purity.

### Example 4

### Evaluation of Activity and Stability of PTP Active Domain

### <4-1> Measurement of Activity Using DiFMUP

The activity of PTP active domain obtained in Example 2 was measured by using DIFMUP (Molecular probe, USA).

10 mM DiFMUP (Molecular probe, USA) suspension was diluted with reaction buffer (20 mM Tris-HCl, pH8.0, 0.01% Triton X-100, 5 mM DTT; Sigma, USA). 10 μM of the substrate (final concentrations are shown in Table 3) was reacted with the PTP active domain obtained in Example 2 at room temperature for 90 minutes. The reaction was terminated by adding 1 mM sodium orthovanadate (Sigma, USA). Relative fluorescence unit (RFU) was calculated by measuring OD₃₅₅/₄₆₀ with victor²1420 multilabel counter plate reader (Perkin Elmer, USA) at a regular time interval for 90 minute reaction. The value was compared with that of substrate alone to evaluate the activity.

TABLE 3
Final concentrations (nM) of reacted PTP active
domain

| | Final | | Final | | Final |
|---|---|---|---|---|---|
| PTP | conc. | PTP | conc. | PTP | conc. |
| T4 | 7.69 | pk18 | 500 | p26 | 526 |
| T7 | 1.35 | T10 | 17.24 | T29 | 1219 |
| T48 | 0.74 | T22 | 1.47 | T40 | 13.15 |
| T8 | 8.06 | T20 | 16.13 | T2 | 658 |
| T23 | 1.61 | PTP1B | 1.43 | pk4 | 588 |
| T39 | 7.69 | T25 | 1.11 | pk7 | 625 |
| T5 | 7.14 | T41 | 11.36 | pk9 | 781 |
| T38 | 161 | T18 | 7.35 | pk10 | 625 |
| T12 | 1282 | pk32 | 1.47 | T33 | 882 |
| T15 | 1.16 | pk28 | 83.3 | pk12 | 1178 |
| pk6 | 75 | T32 | 5.55 | pk13 | 117 |
| pk14 | 625 | p12 | 52 | T27 | 526 |
| pk15 | 1351 | pk17 | 2500 | T46 | 277 |
| pk33 | 9522 | p16 | 156 | pk1 | 108 |
| p44 | 909 | T16 | 2083 | T47 | 91 |
| p21 | 147 | p18 | 588 | T45 | 543 |
| pk35 | 119 | NE3 | 580 | pk8 | 1250 |
| NE1 | 300 | pk3 | 2631 | | |
| p19 | 119 | p49 | 2941 | | |

As a result, as shown in reaction saturation curve in FIG. 7, the purified PTP showed substrate-degrading capacity, which is the property of a normal enzyme, and demonstrated reaction saturation over the time. And, the reaction saturation was accomplished within 20-30 minutes, suggesting that this period of time is favorable for the screening of an inhibitor.

<4-2> Evaluation of Activity after Storing at Room Temperature and at Low Temperature

The stability of the PTP active domain obtained in Example 2 was measured.

PTP active domain was stored at different temperatures including room temperature and low temperature (4° C.) and at different concentrations and for different periods of time, and then the activity was measured by the same manner as described in Example <4-1>, which was compared with that measured in Example <4-1>. The concentration of the reactant protein and reaction time varies from a substrate, but generally the concentration of the protein herein was determined as much as all substrates were not turned into reactants, and as shown in FIG. 7, reaction conditions were regulated for the said concentration of the protein to produce no more reactants from the reaction with the substrate, which was approximately 20-30 minutes.

As a result, the activity was maintained for approximately 6 hours at room temperature. When the domain was stored at a low temperature at the concentration of 0.5˜1.0 mg/ml, the activity was maintained for about 2 weeks.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A method for preparing a recombinant PTP active domain comprising the following steps:
1) investigating homology among subgroups of protein tyrosine phosphatase (PTP) and selecting a region exhibiting high homology;
2) examining whether the selected region of step 1) corresponds to an active domain of a standard protein whose secondary and tertiary structures have already been identified;
3) analyzing the secondary structure of the selected region of step 1) if it corresponds to the active domain and then determining-a boundary of PTP active domain by the location not containing helix or sheet of the secondary structure;
4) determining 2-3 amino acids of the boundary of N-terminal and C-terminal of the PTP active domain primarily determined in step 3) to be small amino acid or the amino acid having electric charge by amino acid analysis;
5) constructing an expression vector containing a polynucleotide encoding the amino acids included in the inside of the boundary of the PTP active domain determined in step 4);
6) generating a transformant by introducing the expression vector of step 5) into a host cell; and,
7) inducing expression of the recombinant PTP active domain by culturing the transformant of step 6) and obtaining the recombinant PTP active domain produced therefrom.

2. The method according to claim 1, wherein the subgroup is composed of receptor, non-receptor, MKP (mitogen-activated protein kinase phosphatase), DUSP (dual-specificity phosphatases) and CDC14 (cell division cycle 14) homologues.

3. The method according to claim 1, wherein the investigation of homology of step 1) is performed by one or more programs selected from the group consisting of ClustalX, KALIGN, MAFFT and Muscle.

4. The method according to claim 1, wherein the secondary structure analysis of step 3) is performed by one or more programs selected from the group consisting of GOR IV SECONDARY STRUCTURE PREDICTION METHOD, PHDsec and Jpred.

5.-6. (canceled)

7. The method according to claim 1, wherein the small amino acid is serine or glycine.

8. The method according to claim 1, wherein the amino acid having electric charge is selected from the group consisting of lysine, arginine, glutamine, asparagine, glutamic acid and aspartic acid.

9. The method according to claim 1, wherein the method additionally includes the step of re-designing the boundary of PTP active domain by treating with protease when the recombinant PTP active domain has low activity and stability.

10. (canceled)

11. The method according to claim 1, wherein the obtaining of the recombinant PTP active domain of step 7) is performed under oxidation-reduction condition.

12. The method according to claim 11, wherein the oxidation-reduction condition is performed by using 5-20 mM DTT or beta-mercaptoethanol.

13. A recombinant PTP active domain represented by the amino acid sequence selected from the group consisting of the amino acid sequences represented by SEQ. ID. NO: 113-SEQ. ID. NO: 135 and SEQ. ID. NO: 137-SEQ. ID. NO: 168.

14.-17. (canceled)

18. A kit for screening PTP inhibitor or activator containing a recombinant PTP active domain represented by the amino acid sequence selected from the group consisting of the amino acid sequences represented by SEQ. ID. NO: 113-SEQ. ID. NO: 135 and SEQ. ID. NO: 137-SEQ. ID. NO: 168.

19. The screening kit according to claim 18, wherein the kit additionally includes a substrate for measuring the activity of PTP active domain, a reaction buffer and a reaction termination reagent.

20. The screening kit according to claim 19, wherein the substrate is selected from the group consisting of DiFMUP (6,8-difluoro-4-methylumbelliferyl phosphate), OMFP (3-O-methylfluorescein phosphate) and PTP substrate peptide labeled with fluorescent material.

21. (canceled)

22. A method for screening PTP activity inhibitor or activator comprising the following steps:
1) treating PTP specific substrate and candidates to the PTP active domain represented by the amino acid sequence selected from the group consisting of the amino acid sequences represented by SEQ. ID. NO: 113-SEQ. ID. NO: 135 and SEQ. ID. NO: 137-SEQ. ID. NO: 168, followed by determining activity based on optical density after measuring the optical density; and,
2) selecting candidates which reduce or increase the activity of the recombinant PTP active domain by comparing the activity of step 1) with that of the non-treated control.

23.-28. (canceled)


SEQUENCE LISTING (179 sequences)

SEQ ID NO: 1 - DNA, 36 bp, Artificial Sequence
  T4 Forward primer
cgcgacgctagcatggcagacgacaataagctcttc

SEQ ID NO: 2 - DNA, 35 bp, Artificial Sequence
  T4 Reverse primer
gctgcgaagctttacttgaagttggcataatctga

SEQ ID NO: 3 - DNA, 36 bp, Artificial Sequence
  T7 Forward primer
ggcacccatatgctagtggctgttgttgccttattg

SEQ ID NO: 4 - DNA, 33 bp, Artificial Sequence
  T7 Reverse primer
gcgggatcctcaatgccttgaatagactggatc

SEQ ID NO: 5 - DNA, 33 bp, Artificial Sequence
  T48 Forward primer
gccccacatatgcgagaccacccacccatcccc

SEQ ID NO: 6 - DNA, 36 bp, Artificial Sequence
  T48 Reverse primer
ggaagatctctacgttgcatagtggtcaaagctgcc

SEQ ID NO: 7 - DNA, 37 bp, Artificial Sequence
  T8 Forward primer
gcgccatatggcagacaagtaccagcaactctccctg

SEQ ID NO: 8 - DNA, 37 bp, Artificial Sequence
  T8 Reverse primer
gcgcggatccctcggctggggcctgggctgactgttg

SEQ ID NO: 9 - DNA, 36 bp, Artificial Sequence
  T23 Forward primer
ccgttacatatggtggagaattttgaggcctacttc

SEQ ID NO: 10 - DNA, 35 bp, Artificial Sequence
  T23 Reverse primer
cccgaattcttaggcgatgtaaccattggtctttc

SEQ ID NO: 11 - DNA, 36 bp, Artificial Sequence
  T39 Forward primer
cacattgctagcatgaagacatcagacagctatggg

SEQ ID NO: 12 - DNA, 36 bp, Artificial Sequence
  T39 Reverse primer
cggctcaagcttctaagatgattccaggtactccaa

SEQ ID NO: 13 - DNA, 33 bp, Artificial Sequence
  T5 Forward primer
gcccaccatatggccagcgataccagcagcctg

SEQ ID NO: 14 - DNA, 33 bp, Artificial Sequence
  T5 Reverse primer
gcgagatcttcagccagaattcaagtattccag

SEQ ID NO: 15 - DNA, 36 bp, Artificial Sequence
  T38 Forward primer
gaccggcatatgcttgccaaggagtggcaggccctc

SEQ ID NO: 16 - DNA, 33 bp, Artificial Sequence
  T38 Reverse primer
ccgggatcctcactggggcagggccttgaggat

SEQ ID NO: 17 - DNA, 33 bp, Artificial Sequence
  T12 Forward primer
cgccagcatatggccacgcggccaccagaccga

SEQ ID NO: 18 - DNA, 33 bp, Artificial Sequence
  T12 Reverse primer
gcgggatcctcactggggaagggccttgaggat

SEQ ID NO: 19 - DNA, 36 bp, Artificial Sequence
  T15 Forward primer
gagcatgctagcatggctagggagtgtggagctggt

SEQ ID NO: 20 - DNA, 36 bp, Artificial Sequence
  T15 Reverse primer
gcgggatccctaggacttgctaacattctcgtatat

SEQ ID NO: 21 - DNA, 38 bp, Artificial Sequence
  T10 Forward primer
cctttccatatgaagcccataggacttcaagagagaag

SEQ ID NO: 22 - DNA, 38 bp, Artificial Sequence
  T10 Reverse primer
gacagtaagctttcaaagtctgctctcatacaggcaca

SEQ ID NO: 23 - DNA, 33 bp, Artificial Sequence
  T22 Forward primer
cgcgaacatatgcttagccacccgccaattccc

SEQ ID NO: 24 - DNA, 36 bp, Artificial Sequence
  T22 Reverse primer
ggcggatcctcagcccacggcctccagcagggcctc

SEQ ID NO: 25 - DNA, 30 bp, Artificial Sequence
  T20 Forward primer
ttcgctagcgccatccgggtggctgacttg

SEQ ID NO: 26 - DNA, 38 bp, Artificial Sequence
  T20 Reverse primer
gcgggatccctaaaaggagcttaaatattccagtgcca

SEQ ID NO: 27 - DNA, 30 bp, Artificial Sequence
  PTP1B Forward primer
atggagatggaaaaggagttcgagcagatc

SEQ ID NO: 28 - DNA, 30 bp, Artificial Sequence
  PTP1B Reverse primer
gtcaacatgtgcgtggctacggtcctcacg

SEQ ID NO: 29 - DNA, 33 bp, Artificial Sequence
  T25 Forward primer
gctcccgctagcatgcccaccatcgagcgggag

SEQ ID NO: 30 - DNA, 33 bp, Artificial Sequence
  T25 Reverse primer
cgcggatccttaggtgtctgtcaatcttggcct

SEQ ID NO: 31 - DNA, 33 bp, Artificial Sequence
  T41 Forward primer
tcagagcatatggaggagaagatcgaggatgac

SEQ ID NO: 32 - DNA, 36 bp, Artificial Sequence
  T41 Reverse primer
gtggacgctagcatgaaatatttgggcagtcccatt

SEQ ID NO: 33 - DNA, 33 bp, Artificial Sequence
  T18 Forward primer
gccccccatatggtgaggtggtttcaccgagac

SEQ ID NO: 34 - DNA, 34 bp, Artificial Sequence
  T18 Reverse primer
ccggaattctcacttcctcttgagggaacccttg

SEQ ID NO: 35 - DNA, 36 bp, Artificial Sequence
  pk32 Forward primer
gaaccccatatgtctgtgaacacaccccgggaggtc

SEQ ID NO: 36 - DNA, 32 bp, Artificial Sequence
  pk32 Reverse primer
cgggatcctcaggggctgggttcctcaggcag

SEQ ID NO: 37 - DNA, 33 bp, Artificial Sequence
  pk28 Forward primer
ccgcggcatatggaacatcacgggcaattaaaa

SEQ ID NO: 38 - DNA, 32 bp, Artificial Sequence
  pk28 Reverse primer
cgggatcctcacctgcagtgcaccacgaccgg

SEQ ID NO: 39 - DNA, 33 bp, Artificial Sequence
  T32 Forward primer
gcagtacatatgaatgggaagttatcagaagag

SEQ ID NO: 40 - DNA, 38 bp, Artificial Sequence
  T32 Reverse primer
ggcggatcctcacttcagaagctgaggctgctgttttt

SEQ ID NO: 41 - DNA, 33 bp, Artificial Sequence
  T40 Forward primer
gagcagcatatggcaggcctggaggcacagaag

SEQ ID NO: 42 - DNA, 33 bp, Artificial Sequence
  T40 Reverse primer
cgcggatccttaaatgagtctggagttttggag

SEQ ID NO: 43 - DNA, 33 bp, Artificial Sequence
  T2 Forward primer
ctagggcatatgaaaaagactcgagtagatgca

SEQ ID NO: 44 - DNA, 33 bp, Artificial Sequence
  T2 Reverse primer
cgcggatccttagatgagcctggagcttttcag

SEQ ID NO: 45 - DNA, 33 bp, Artificial Sequence
  pk4 Forward primer
aggccgcatatggtcatggaagtgggcaccctg

SEQ ID NO: 46 - DNA, 34 bp, Artificial Sequence
  pk4 Reverse primer
ggcggatcctcagctcccagcctctgccgaacag

SEQ ID NO: 47 - DNA, 30 bp, Artificial Sequence
  pk7 Forward primer
gttcatatgagtgccacagagcccttggac

SEQ ID NO: 48 - DNA, 36 bp, Artificial Sequence
  pk7 Reverse primer
gcgggatcctcaggacgtggccagcacctgggactc

SEQ ID NO: 49 - DNA, 36 bp, Artificial Sequence
  pk8 Forward primer
gcggaccatatgggcccagttgaaatccttcccttc

SEQ ID NO: 50 - DNA, 37 bp, Artificial Sequence
  pk8 Reverse primer
gcgagatcttcacgtggagggcaggatctcagattcg

SEQ ID NO: 51 - DNA, 36 bp, Artificial Sequence
  pk9 Forward primer
ggcagccatatgtccttcccagtggagatcttgccc

SEQ ID NO: 52 - DNA, 36 bp, Artificial Sequence
  pk9 Reverse primer
cgcggatcctcagctgagtcccagcgtcctctcgaa

SEQ ID NO: 53 - DNA, 33 bp, Artificial Sequence
  pk10 Forward primer
gctggccatatgttgcgccgcctgcgcaagggc

SEQ ID NO: 54 - DNA, 32 bp, Artificial Sequence
  pk10 Reverse primer
cgggatcctcacgtggactccagcgtattgag

SEQ ID NO: 55 - DNA, 33 bp, Artificial Sequence
  T33 Forward primer
tgcccccatatggctggggaccggctcccgagg

SEQ ID NO: 56 - DNA, 36 bp, Artificial Sequence
  T33 Reverse primer
gcgggatcctcatgagggggtgcccgggtcgccctg

SEQ ID NO: 57 - DNA, 32 bp, Artificial Sequence
  pk12 Forward primer
cgatcgcatatggagggtctgggccgctcgtg

SEQ ID NO: 58 - DNA, 31 bp, Artificial Sequence
  pk12 Reverse primer
cgggatccctaggtgggggccagctcgaagg

SEQ ID NO: 59 - DNA, 33 bp, Artificial Sequence
  pk13 Forward primer
ctggaccatatgcagcggctgaacatcggctac

SEQ ID NO: 60 - DNA, 33 bp, Artificial Sequence
  pk13 Reverse primer
cgggatcctcacacaaccgtctccactcccatc

SEQ ID NO: 61 - DNA, 31 bp, Artificial Sequence
  T27 Forward primer
gttgcccatatggggccaacccgaattcttc

SEQ ID NO: 62 - DNA, 28 bp, Artificial Sequence
  T27 Reverse primer
ggatccttatgatgctccagtctggttc

SEQ ID NO: 63 - DNA, 33 bp, Artificial Sequence
  pk6 Forward primer
gccgcccatatgtcgggctcgttcgagctctcg

SEQ ID NO: 64 - DNA, 30 bp, Artificial Sequence
  pk6 Reverse primer
cgggatccctagggtttcaacttcccctcc

SEQ ID NO: 65 - DNA, 36 bp, Artificial Sequence
  pk14 Forward primer
gccaagcatatgggcggaaaccacatccccgaaagg

SEQ ID NO: 66 - DNA, 36 bp, Artificial Sequence
  pk14 Reverse primer
gcgggatcctcaggaattccaattctttctgatagg

SEQ ID NO: 67 - DNA, 36 bp, Artificial Sequence
  pk15 Forward primer
agcgcccatatggtcagctgtgccgggcagatgctg

SEQ ID NO: 68 - DNA, 32 bp, Artificial Sequence
  pk15 Reverse primer
cgggatcctcatatttttcctgtttgtgatcc

SEQ ID NO: 69 - DNA, 33 bp, Artificial Sequence
  pk33 Forward primer
ggctggcatatggctgagacctctctcccagag

SEQ ID NO: 70 - DNA, 30 bp, Artificial Sequence
  pk33 Reverse primer
cgggatcctcagctctggccggcaccccgc

SEQ ID NO: 71 - DNA, 33 bp, Artificial Sequence
  p44 Forward primer
tcccaccatatggactcactgcagaagcaggac

SEQ ID NO: 72 - DNA, 24 bp, Artificial Sequence
  p44 Reverse primer
gccaagggtcagggatcctggctg

SEQ ID NO: 73 - DNA, 34 bp, Artificial Sequence
  p21 Forward primer
cccgggcatatgggcaatggcatgaccaaggtac

SEQ ID NO: 74 - DNA, 33 bp, Artificial Sequence
  p21 Reverse primer
gcgggatcctcacttgccgcccttgcgggacag

SEQ ID NO: 75 - DNA, 33 bp, Artificial Sequence; identical to SEQ ID NO: 74
  pk35 Forward primer

SEQ ID NO: 76 - DNA, 32 bp, Artificial Sequence
  pk35 Reverse primer
cgggatcctcacagtggaatcatcaaacggac

SEQ ID NO: 77 - DNA, 30 bp, Artificial Sequence
  NE1 Forward primer
ccaggggctagccgctaactggaaagaaaa

SEQ ID NO: 78 - DNA, 30 bp, Artificial Sequence
  NE1 Reverse primer
gtcggatccttagctttctttgccctcttg

SEQ ID NO: 79 - DNA, 26 bp, Artificial Sequence
  p19 Forward primer
atgacagcatccgcgtcctccttttc

SEQ ID NO: 80 - DNA, 24 bp, Artificial Sequence
  p19 Reverse primer
ttacattgatatcatcatacgtag

SEQ ID NO: 81 - DNA, 33 bp, Artificial Sequence
  pk18 Forward primer
gcagcccatatggggaatgggatgaacaagatc

SEQ ID NO: 82 - DNA, 33 bp, Artificial Sequence
  pk18 Reverse primer
cgggatccttacagtcttctgagaaaggcccag

SEQ ID NO: 83 - DNA, 33 bp, Artificial Sequence
  p12 Forward primer
gggaagcatatgggtcgggcgcaccgggactgg

SEQ ID NO: 84 - DNA, 38 bp, Artificial Sequence
  p12 Reverse primer
ggcaccaagctttcagaactctttaagaacatccagct

SEQ ID NO: 85 - DNA, 34 bp, Artificial Sequence
  pk17 Forward primer
ctggagcatatgccaaccgttcaacatcctttcc

SEQ ID NO: 86 - DNA, 34 bp, Artificial Sequence
  pk17 Reverse primer
gcgggatcctcatgcttccagaccctgccgcagc

SEQ ID NO: 87 - DNA, 36 bp, Artificial Sequence
  p16 Forward primer
gcggcggctagcatgggcgtgcagccccccaacttc

SEQ ID NO: 88 - DNA, 35 bp, Artificial Sequence
  p16 Reverse primer
cgcgcctcgagtttcgttcgctggtagaactggaa

SEQ ID NO: 89 - DNA, 36 bp, Artificial Sequence
  T16 Forward primer
ggcggcgctagcatggctcacaacaagatcccgccg

SEQ ID NO: 90 - DNA, 36 bp, Artificial Sequence
  T16 Reverse primer
tgaggatccttatgattccttctttccatcctcatc

SEQ ID NO: 91 - DNA, 33 bp, Artificial Sequence
  p18 Forward primer
ccgggacatatggacaagccctcccttatcttc

SEQ ID NO: 92 - DNA, 33 bp, Artificial Sequence
  p18 Reverse primer
gcgggatcctcagcttgcatccaagatgccttc

SEQ ID NO: 93 - DNA, 34 bp, Artificial Sequence
  NE3 Forward primer
cttggtcatatggatagccctacacagatatttg

SEQ ID NO: 94 - DNA, 33 bp, Artificial Sequence
  NE3 Reverse primer
gcgggatcctcaccttgccagcaagatcccctg

SEQ ID NO: 95 - DNA, 33 bp, Artificial Sequence
  pk3 Forward primer
gcggctcatatgaaccgcccagctcctgtggaa

SEQ ID NO: 96 - DNA, 36 bp, Artificial Sequence
  pk3 Reverse primer
gcgggatcctcaggaatctttgaaacgcagccgcat

SEQ ID NO: 97 - DNA, 36 bp, Artificial Sequence
  p49 Forward primer
cgccgagctagcatgcgttttctgataactcacaac

SEQ ID NO: 98 - DNA, 33 bp, Artificial Sequence
  p49 Reverse primer
cgggatccctactgaacacagcaatgcccattg

SEQ ID NO: 99 - DNA, 34 bp, Artificial Sequence
  p26 Forward primer
gcgacccatatggccccggtggaggtgagctaca

SEQ ID NO: 100 - DNA, 37 bp, Artificial Sequence
  p26 Reverse primer
cgcggatcctcaggtcttgtgcgtgtgtgggtctttg

SEQ ID NO: 101 - DNA, 36 bp, Artificial Sequence
  T29 Forward primer
ggcggccatatgtcgtcgacctcgccgggtgtgaag

SEQ ID NO: 102 - DNA, 38 bp, Artificial Sequence
  T29 Reverse primer
gccggatccttatttggagaaggctgctctgtgttgtc

SEQ ID NO: 103 - DNA, 27 bp, Artificial Sequence
  T46 Forward primer
atggcggaacaggctaccaagtccgtg

SEQ ID NO: 104 - DNA, 29 bp, Artificial Sequence
  T46 Reverse primer
tcagtgggccttctccaagaacgctctgc

SEQ ID NO: 105 - DNA, 31 bp, Artificial Sequence
  pk1 Forward primer
gctctagacttataggagacttctccaaggg

SEQ ID NO: 106 - DNA, 33 bp, Artificial Sequence
  pk1 Reverse primer
gccctaggtcagagcttcttcagacgactgtac

SEQ ID NO: 107 - DNA, 36 bp, Artificial Sequence
  T47 Forward primer
gaccaccatatgctgattggagattactctaaggcc

SEQ ID NO: 108 - DNA, 33 bp, Artificial Sequence
  T47 Reverse primer
ccgggatcctcactggtcctgcagccggctaca

SEQ ID NO: 109 - DNA, 36 bp, Artificial Sequence
  T45 Forward primer
gattctgctagcgggcacctgattggtgatttttcc

SEQ ID NO: 110 - DNA, 33 bp, Artificial Sequence
  T45 Reverse primer
ccgggatcctcatgggctcatgtccttcaccag

SEQ ID NO: 111 - DNA, 33 bp, Artificial Sequence
  Eya2 Forward primer
gacaatcatatggagcgtgtgttcgtgtgggac

SEQ ID NO: 112 - DNA, 33 bp, Artificial Sequence
  Eya2 Reverse primer
gaattcttataaatactccagctccagggcgtg

SEQ ID NO: 113 - protein, 569 aa, Artificial Sequence
  Amino acid sequence of PTP active domain T4
MADDNKLFREEFNALPACPIQATCEAASKEENKEKNRYVNILPYDHSRVHLTPVEGVPDSDYINASFINGYQEKNKFIAAQGPKEETVNDFWRMIWEQNTATIVMVTNLKERKECKCAQYWPDQGCWTYGNIRVSVEDVTVLVDYTVRKFCIQQVGDMTNRKPQRLITQFHFTSWPDFGVPFTPIGMLKFLKKVKACNPQYAGAIVVHCSAGVGRTGTFVVIDAMLDMMHTERKVDVYGFVSRIRAQRCQMVQTDMQYVFIYQALLEHYLYGDTELEVTSLETHLQKIYNKIPGTSNNGLEEEFKKLTSIKIQNDKMRTGNLPANMKKNRVLQIIPYEFNRVIIPVKRGEENTDYVNASFIDGYRQKDSYIASQGPLLHTIEDFWRMIWEWKSCSIVMLTELEERGQEKCAQYWPSDGLVSYGDITVELKKEEECESYTVRDLLVTNTRENKSRQIRQFHFHGWPEVGIPSDGKGMISIIAAVQKQQQQSGNHPITVHCSAGAGRTGTFCALSTVLERVKAEGILDVFQTVKSLRLQRPHMVQTLEQYEFCYKVVQEYIDAFSDYANFK

SEQ ID NO: 114 - protein, 284 aa, Artificial Sequence
  Amino acid sequence of PTP active domain T7
KINQFEGHFMKLQADSNYLLSKEYEELKDVGRNQSCDIALLPENRGKNRYNNILPYDATRVKLSNVDDDPCSDYINASYIPGNNFRREYIVTQGPLPGTKDDFWKMVWEQNVHNIVMVTQCVEKGRVKCDHYWPADQDSLYYGDLILQMLSESVLPEWTIREFKICGEEQLDAHRLIRHFHYTVWPDHGVPETTQSLIQFVRTVRDYINRSPGAGPTVVHCSAGVGRTGTFIALDRILQQLDSKDSVDIYGAVHDLRLHRVHMVQTECQYVYLHQCVRDVLRAR

SEQ ID NO: 115 - protein, 582 aa, Artificial Sequence
  Amino acid sequence of PTP active domain T48
GRIVYGLRPGRSYQFNVKTVSGDSWKTYSKPIFGSVRTKPDKIQNLHCRPQNSTAIACSWIPPDSDFDGYSIECRKMDTQEVEFSRKLEKEKSLLNIMMLVPHKRYLVSIKVQSAGMTSEVVEDSTITMIDRPPPPPPHIRVNEKDVLISKSSINFTVNCSWFSDTNGAVKYFTVVVREADGSDELKPEQQHPLPSYLEYRHNASIRVYQTNYFASKCAENPNSNSKSFNIKLGAEMESLGGKRDPTQQKFCDGPLKPHTAYRISIRAFTQLFDEDLKEFTKPLYSDTFFSLPITTESEPLFGAIEGVSAGLFLIGMLVAVVALLICRQKVSHGRERPSARLSIRRDRPLSVHLNLGQKGNRKTSCPIKINQFEGHFMKLQADSNYLLSKEYEELKDVGRNQSCDIALLPENRGKNRYNNILPYDATRVKLSNVDDDPCSDYINASYIPGNNFRREYIVTQGPLPGTKDDFWKMVWEQNVHNIVMVTQCVEKGRVKCDHYWPADQDSLYYGDLILQMLSESVLPEWTIREFKICGEEQLDAHRLIRHFHYTVWPDHGVPETTQSLIQFVRTVRDYINRSPGA

SEQ ID NO: 116 - protein, 269 aa, Artificial Sequence
  Amino acid sequence of PTP active domain T8
PVKDLTLRNRSTEDLHVTWSGANGDVDQYEIQLLFNDMKVFPPFHLVNTATEYRFTSLTPGRQYKILVLTISGDVQQSAFIEGFTVPSAVKNIHISPNGATDSLTVNWTPGGGDVDSYTVSAFRHSQKVDSQTIPKHVFEHTFHRLEAGEQYQIMIASVSGSLKNQINVVGRTVPASVQGVIADNAYSSYSLIVSWQKAAGVAERYDILLLTENGILLRNTSEPATTKQHKFEDLTPGKKYKIQILTVSGGLFSKEAQTEGRTVPAAVT

SEQ ID NO: 117 - protein, 312 aa, Artificial Sequence
  Amino acid sequence of PTP active domain T23
ENFEAYFKKQQADSNCGFAEEYEDLKLVGISQPKYAAELAENRGKNRYNNVLPYDISRVKLSVQTHSTDDYINANYMPGYHSKKDFIATQGPLPNTLKDFWRMVWEKNVYAIIMLTKCVEQGRTKCEEYWPSKQAQDYGDITVAMTSEIVLPEWTIRDFTVKNIQTSESHPLRQFHFTSWPDHGVPDTTDLLINFRYLVRDYMKQSPPESPILVHCSAGVGRTGTFIAIDRLIYQIENENTVDVYGIVYDLRMHRPLMVQTEDQYVFLNQCVLDIVRSQKDSKVDLIYQNTTAMTIYENLAPVTTFGKTNGY

SEQ ID NO: 118 - protein, 561 aa, Artificial Sequence
  Amino acid sequence of PTP active domain T39
MKTSDSYGFKEEYESFFEGQSASWDVAKKDQNRAKNRYGNIIAYDHSRVILQPVEDDPSSDYINANYIDGYQRPSHYIATQGPVHETVYDFWRMIWQEQSACIVMVTNLVEVGRVKCYKYWPDDTEVYGDFKVTCVEMEPLAEYVVRTFTLERRGYNEIREVKQFHFTGWPDHGVPYHATGLLSFIRRVKLSNPPSAGPIVVHCSAGAGRTGCYIVIDIMLDMAEREGVVDIYNCVKALRSRRINMVQTEEQYIFIHDAILEACLCGETAIPVCEFKAAYFDMIRIDSQTNSSHLKDEFQTLNSVTPRLQAEDCSIACLPRNHDKNRFMDMLPPDRCLPFLITIDGESSNYINAALMDSYRQPAAFIVTQYPLPNTVKDFWRLVYDYGCTSIVMLNEVDLSQGCPQYWPEEGMLRYGPIQVECMSCSMDCDVINRIFRICNLTRPQEGYLMVQQFQYLGWASHREVPGSKRSFLKLILQVEKWQEECEEGEGRTIIHCLNGGGRSGMFCAIGIVVEMVKRQNVVDVFHAVKTLRNSKPNMVEAPEQYRFCYDVALEYLESS

SEQ ID NO: 119 - protein, 605 aa, Artificial Sequence
  Amino acid sequence of PTP active domain T5
MASDTSSLVQSHTYKKREPADVPYQTGQLHPAIRVADLLQHITQMKCAEGYGFKEEYESFFEGQSAPWDSAKKDENRMKNRYGNIIAYDHSRVRLQTIEGDTNSDYINGNYIDGYHRPNHYIATQGPMQETIYDFWRMVWHENTASIIMVTNLVEVGRVKCCKYWPDDTEIYKDIKVTLIETELLAEYVIRTFAVEKRGVHEIREIRQFHFTGWPDHGVPYHATGLLGFVRQVKSKSPPSAGPLVVHCSAGAGRTGCFIVIDIMLDMAEREGVVDIYNCVRELRSRRVNMVQTEEQYVFIHDAILEACLCGDTSVPASQVRSLYYDMNKLDPQTNSSQIKEEFRTLNMVTPTLRVEDCSIALLPRNHEKNRCMDILPPDRCLPFLITIDGESSNYINAALMDSYKQPSAFIVTQHPLPNTVKDFWRLVLDYHCTSVVMLNDVDPAQLCPQYWPENGVHRHGPIQVEFVSADLEEDIISRIFRIYNAARPQDGYRMVQQFQFLGWPMYRDTPVSKRSFLKLIRQVDKWQEEYNGGEGPTVVHCLNGGGRSGTFCAISIVCEMLRHQRTVDVFHAVKTLRNNKPNMVDLLDQYKFCYEVALEYLNSG

SEQ ID NO: 120 - protein, 270 aa, Artificial Sequence
  Amino acid sequence of PTP active domain T38
LAKEWQALCAYQAEPNTCATAQGEGNIKKNRHPDFLPYDHARIKLKVESSPSRSDYINASPIIEHDPRMPAYIATQGPLSHTIADFWQMVWESGCTVIVMLTPLVEDGVKQCDRYWPDEGASLYHVYEVNLVSEHIWCEDFLVRSFYLKNVQTQETRTLTQFHFLSWPAEGTPASTRPLLDFRRKVNKCYRGRSCPIIVHCSDGAGRTGTYILIDMVLNRMAKGVKEIDIAATLEHVRDQRPGLVRSKDQFEFALTAVAEEVNAILKALP

SEQ ID NO: 121 - protein, 342 aa, Artificial Sequence [sequence omitted]

SEQ ID NO: 122 - protein, 366 aa, Artificial Sequence
  Amino acid sequence of PTP active domain T15
MARECGAGTFVNFASLERDGKLPYNWRRSIFAFLTLLPSCLWTDYLLAFYINPWSKNGLKKRKLTNPVQLDDFDAYIKDMAKDSDYKFSLQFEELKLIGLDIPHFAADLPLNRCKNRYTNILPYDFSRVRLVSMNEEEGADYINANYIPGYNSPQEYIATQGPLPETRNDFWKMVLQQKSQIIVMLTQCNEKRRVKCDHYWPFTEEPIAYGDITVEMISEEEQDDWACRHFRINYADEMQDVMHFNYTAWPDHGVPTANAAESILQFVHMVRQQATKSKGPMIIHCSAGVGRTGTFIALDRLLQHIRDHEFVDILGLVSEMRSYRMSMVQTEEQYIFIHQCVQLMWMKKKQQFCISDVIYENVSKS

SEQ ID NO: 123 - protein, 322 aa, Artificial Sequence
  Amino acid sequence of PTP active domain T10
MKPIGLQERRGSNVSLTLDMSSLGNIEPFVSIPTPREKVAMEYLQSASRILTRSQLRDVVASSHLLQSEFMEIPMNFVDPKEIDIPRHGTKNRYKTILPNPLSRVCLRPKNVTDSLSTYINANYIRGYSGKEKAFIATQGPMINTVDDFWQMVWQEDSPVIVMITKLKEKNEKCVLYWPEKRGIYGKVEVLVISVNECDNYTIRNLVLKQGSHTQHVKHYWYTSWPDHKTPDSAQPLLQLMLDVEEDRLASQGRGPVVVHCSAGIGRTGCFIATSIGCQQLKEEGVVDALSIVCQLRMDRGGMVQTSEQYEFVHHALCLYES

SEQ ID NO: 124 - protein, 284 aa, Artificial Sequence
  Amino acid sequence of PTP active domain T22
MLSHPPIPIADMAEHTERLKANDSLKLSQEYESIDPGQQFTWEHSNLEVNKPKNRYANVIAYDHSRVILQPIEGIMGSDYINANYVDGYRRQNAYIATQGPLPETFGDFWRMVWEQRSATIVMMTRLEEKSRIKCDQYWPNRGTETYGFIQVTLLDTIELATFCVRTFSLHKNGSSEKREVRQFQFTAWPDHGVPEYPTPFLAFLRRVKTCNPPDAGPIVVHCSAGVGRTGCFIVIDAMLERIKPEKTVDVYGHVTLMRSQRNYMVQTEDQYSFIHEALLEAVG

SEQ ID NO: 125 - protein, 291 aa, Artificial Sequence
  Amino acid sequence of PTP active domain T20
AIRVADLLQHITQMKRGQGYGFKEEYEALPEGQTASWDTAKEDENRNKNRYGNIISYDHSRVRLLVLDGDPHSDYINANYIDGYHRPRHYIATQGPMQETVKDFWRMIWQENSASIVMVTNLVEVGRVKCVRYWPDDTEVYGDIKVTLIETEPLAEYVIRTFTVQKKGYHEIRELRLFHFTSWPDHGVPCYATGLLGFVRQVKFLNPPEAGPIVVHCSAGAGRTGCFIAIDTMLDMAENEGVVDIFNCVRELRAQRVNLVQTEEQYVFVHDAILEACLCGNTAIPVCEFRS

SEQ ID NO: 126 - protein, 299 aa, Artificial Sequence
  Amino acid sequence of PTP active domain PTP1B
MEMEKEFEQIDKSGSWAAIYQDIRHEASDFPCRVAKLPKNKNRNRYRDVSPFDHSRIKLHQEDNDYINASLIKMEEAQRSYILTQGPLPNTCGHFWEMVWEQKSRGVVMLNRVMEKGSLKCAQYWPQKEEKEMIFEDTNLKLTLISEDIKSYYTVRQLELENLTTQETREILHFHYTTWPDFGVPESPASFLNFLFKVRESGSLSPEHGPVVVHCSAGIGRSGTFCLADTCLLLMDKRKDPSSVDIKKVLLEMRKFRMGLIQTADQLRFSYLAVIEGAKFIMGDSSVQDQWKELSHEDL

SEQ ID NO: 127 - protein, 387 aa, Artificial Sequence
  Amino acid sequence of PTP active domain T25
MPTTIEREFEELDTQRRWQPLYLEIRNESHDYPHRVAKFPENRNRNRYRDVSPYDHSRVKLQNAENDYINASLVDIEEAQRSYILTQGPLPNTCCHFWLMVWQQKTKAVVMLNRIVEKESVKCAQYWPTDDQEMLFKETGFSVKLLSEDVKSYYTVHLLQLENINSGETRTISHFHYTTWPDFGVPESPASFLNFLFKVRESGSLNPDHGPAVIHCSAGIGRSGTFSLVDTCLVLMEKGDDINIKQVLLNMRKYRMGLIQTPDQLRFSYMAIIEGAKCIKGDSSIQKRWKELSKEDLSPAFDHSPNKIMTEKYNGNRIGLEEEKLTGDRCTGLSSKMQDTMEENSESALRKRIREDRKATTAQKVQQMKQRLNENERKRKRPRLTDT

SEQ ID NO: 128 - protein, 381 aa, Artificial Sequence
  Amino acid sequence of PTP active domain T41
VSRQPSFTYSEWMEEKIEDDFLDLDPVPETPVFDCVMDIKPEADPTSLTVKSMGLQERRGSNVSLTLDMCTPGCNEEGFGYLMSPREESAREYLLSASRVLQAEELHEKALDPFLLQAEFFEIPMNFVVPKEYDIPGRCRKNRYKTILPNPHSRVCLTSPDPDDPLSSYINANYIRGYGGEEKVYIATQGPIVSTVADFWRMVWQEHTPIIVMITNIEEMNEKCTEYWPEEQVAYDGVEITVQKVIHTEDYRLRLISLKSGTEERGLKHYWFTSWPDQKTPDRAPPLLHLVREVEEAAQQEGPHCAPIIVHCSAGIGRTGCFIATSICCQQLRQEGVVDILKTTCQLRQDRGGMIQHCEQYQFVHHVMSLYEKQLSHQSPE

SEQ ID NO: 129 - protein, 595 aa, Artificial Sequence
  Amino acid sequence of PTP active domain T18
MVRWFHRDLSGLDAETLLKGRGVHGSFLARPSRKNQGDFSLSVRVGDQVTHIRIQNSGDFYDLYGGEKFATLTELVEYYTQQQGVLQDRDGTIIHLKYPLNCSDPTSERWYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPYYATRVNAADIENRVLELNKKQESEDTAKAGFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQFIETTKKKLEVLQSQKGQESEYGNITYPPAMKNAHAKASRTSSKHKEDVYENLHTKNKREEKVKKQRSADKEKSKGSLKRK

SEQ ID NO: 130 - protein, 298 aa, Artificial Sequence
  Amino acid sequence of PTP active domain pk32
QPPPEKTPAKKHVRLQERRGSNVALMLDVRSLGAVEPICSVNTPREVTLHFLRTAGHPLTRWALQRQPPSPKQLEEEFLKIPSNFVSPEDLDIPGHASKDRYKTILPNPQSRVCLGRAQSQEDGDYINANYIRGYDGKEKVYIATQGPMPNTVSDFWEMVWQEEVSLIVMLTQLREGKEKCVHYWPTEEETYGPFQIRIQDMKECPEYTVRQLTIQYQEERRSVKHILFSAWPDHQTPESAGPLLRLVAEVEESPETAAHPGPIVVHCSAGIGRTGCFIATRIGCQQLKARGEVDILG

SEQ ID NO: 131 - protein, 526 aa, Artificial Sequence
  Amino acid sequence of PTP active domain pk28
MTSRRWFHPNITGVEAENLLLTRGVDGSFLARPSKSNPGDFTLSVRRNGAVTHIKIQNTGDYYDLYGGEKFATLAELVQYYMEHHGQLKEKNGDVIELKYPLNCADPTSERWFHGHLSGKEAEKLLTEKGKHGSFLVRESQSHPGDFVLSVRTGDDKGESNDGKSKVTHVMIRCQELKYDVGGGERFDSLTDLVEHYKKNPMVETLGTVLQLKQPLNTTRINAAEIESRVRELSKLAETTDKVKQGFWEEFETLQQQECKLLYSRKEGQRQENKNKNRYKNILPFDHTRVVLHDGDPNEPVSDYINANIIMPEFETKCNNSKPKKSYIATQGCLQNTVNDFWRMVFQENSRVIVMTTKEVERGKSKCVKYWPDEYALKEYGVMRVRNVKESAAHDYTLRELKLSKVGQGNTERTVWQYHFRTWPDHGVPSDPGGVLDFLEEVHHKQESIMDAGPVVVHCSAGIGRTGTFIVIDILIDIIREKGVDCDIDVPKTIQMVRSQRSGMVQTEAQYRFIYMAVQHYIETLQ

SEQ ID NO: 132 - protein, 396 aa, Artificial Sequence
  Amino acid sequence of PTP active domain T32
MNGKLSEERTEDTDCDGSPLPEYFTEATKMNGCEEYCEEKVKSESLIQKPQEKKTDDDEITWGNDELPIERTNHEDSDKDHSFLTNDELAVLPVVKVLPSGKYTGANLKSVIRVLRGLLDQGIPSKELENLQELKPLDQCLIGQTKENRRKNRYKNILPYDATRVPLGDEGGYINASFIKIPVGKEEFVYIACQGPLPTTVGDFWQMIWEQKSTVIAMMTQEVEGEKIKCQRYWPNILGKTTMVSNRLRLALVRMQQLKGFVVRAMTLEDIQTREVRHISHLNFTAWPDHDTPSQPDDLLTFISYMRHIHRSGPIITHCSAGIGRSGTLICIDVVLGLISQDLDFDISDLVRCMRLQRHGMVQTEDQYIFCYQVILYVLTRLQAEEEQKQQPQLLK

SEQ ID NO: 133 - protein, 322 aa, Artificial Sequence [sequence omitted]

SEQ ID NO: 134 - protein, 336 aa, Artificial Sequence [sequence omitted]

SEQ ID NO: 135 - protein, 151 aa, Artificial Sequence
  Amino acid sequence of PTP active domain pk4
GPVEILPFLYLGSAYHASRKDMLDALGITALINVSANCPNHFEGHYQYKSIPVEDNHKADISSWFNEAIDFIDSIKNAGGRVFVHCQAGISRSATICLAYLMRTNRVKLDEAFEFVKQRRSIISPNFSFMGQLLQFESQVLAPHCSAEAGS

SEQ ID NO: 136 - protein, 165 aa, Artificial Sequence
  Amino acid sequence of PTP active domain pk7
SATEPLDLGCSSCGTPLHDQGGPVEILPFLYLGSAYHAARRDMLDALGITALLNVSSDCPNHFEGHYQYKCIPVEDNHKADISSWFMEAIEYIDAVKDCRGRVLVHCQAGISRSATICLAYLMMKKRVRLEEAFEFVKQRRSIISPNFSFMGQLLQFESQVLATS

SEQ ID NO: 137 - protein, 144 aa, Artificial Sequence
  Amino acid sequence of PTP active domain pk8
GPVEILPFLYLGSAYHASKCEFLANLHITALLNVSRRTSEACATHLHYKWIPVEDSHTADISSHFQEAIDFIDCVREKGGKVLVHCEAGISRSPTICMAYLMKTKQFRLKEAFDYIKQRRSMVSPNFGFMGQLLQYESEILPST

SEQ ID NO: 138 - protein, 144 aa, Artificial Sequence
  Amino acid sequence of PTP active domain pk9
SFPVEILPFLYLGCAKDSTNLDVLEEFGIKYILNVTPNLPNLFENAGEFKYKQIPISDHWSQNLSQFFPEAISFIDEARGKNCGVLVHCLAGISRSVTVTVAYLMQKLNLSMNDAYDIVKMKKSNISPNFNFMGQLLDFERTLG

SEQ ID NO: 139 - protein, 147 aa, Artificial Sequence
  Amino acid sequence of PTP active domain pk10
AFPVQILPYLYLGCAKDSTNLDVLGKYGIKYILNVTPNLPNAFEHGGEFTYKQIPISDHWSQNLSQFFPEAISFIDEARSKKCGVLVHCLAGISRSVTVTVAYLMQKMNLSLNDAYDFVKRKKSNISPNFNFMGQLLDFERTLGLSS

SEQ ID NO: 140 - protein, 153 aa, Artificial Sequence
  Amino acid sequence of PTP active domain T33
GLTRILPHLYLGSQKDVLNKDLMTQNGISYVLNASNSCPKPDFICESRFMRVPINDNYCEKLLPWLDKSIEFIDKAKLSSCQVIVHCLAGISRSATIAIAYIMKTMGMSSDDAYRFVKDRRPSISPNFNFLGQLLEYERTLKLLAALQGDPGT

SEQ ID NO: 141 - protein, 151 aa, Artificial Sequence
  Amino acid sequence of PTP active domain pk12
ASFPVQILPNLYLGSARDSANLESLAKLGIRYILNVTPNLPNFFEKNGDFHYKQIPISDHWSQNLSRFFPEAIEFIDEALSQNCGVLVHCLAGVSRSVTVTVAYLMQKLHLSLNDAYDLVKRKKSNISPNFNFMGQLLDFERSLRLEERHS

SEQ ID NO: 142 - protein, 148 aa, Artificial Sequence
  Amino acid sequence of PTP active domain pk13
AELTPILPFLFLGNEQDAQDLDTMQRLNIGYVINVTTHLPLYHYEKGLFNYKRLPATDSNKQNLRQYFEEAFEFIEEAHQCGKGLLIHCQAGVSRSATIVIAYLMKHTRMTMTDAYKFVKGKRPIISPNLNFMGQLLEFEEDLNNGVT

SEQ ID NO: 143 - protein, 148 aa, Artificial Sequence
  Amino acid sequence of PTP active domain T27
GPTRILPNLYLGCQRDVLNKELMQQNGIGYVLNASNTCPKPDFIPESHFLRVPVNDSFCEKILPWLDKSVDFIEKAKASNGCVLVHCLAGISRSATIAIAYIMKRMDMSLDEAYRFVKEKRPTISPNFNFLGQLLDYEKKIKNQTGAS

SEQ ID NO: 144 - protein, 185 aa, Artificial Sequence
  Amino acid sequence of PTP active domain pk6
MSGSFELSVQDLNDLLSDGSGCYSLPSQPCNEVTPRIYVGNASVAQDIPKLQKLGITHVLNAAEGRSFMHVNTNANFYKDSGITYLGIKANDTQEFNLSAYFERAADFIDQALAQKNGRVLVHCREGYSRSPTLVIAYLMMRQKMDVKSALSIVRQNREIGPNDGFLAQLCQLNDRLAKEGKLKP

SEQ ID NO: 145 - protein, 184 aa, Artificial Sequence
  Amino acid sequence of PTP active domain pk14
GGNHIPERWKDYLPVGQRMPGTRFIAFKVPLQKSFEKKLAPEECFSPLDLFNKIREQNEELGLIIDLTYTQRYYKPEDLPETVPYLKIFTVGHQVPDDETIFKFKHAVNGFLKENKDNDKLIGVHCTHGLNRTGYLICRYLIDVEGVRPDDAIELFNRCRGHCLERQNYIEDLQNGPIRKNWNS

SEQ ID NO: 146 - protein, 320 aa, Artificial Sequence
  Amino acid sequence of PTP active domain pk15
VSCAGQMLEVQPGLYFGGAAAVAEPDHLREAGITAVLTVDSEEPSFKAGPGVEDLWRLFVPALDKPETDLLSHLDRCVAFIGQARAEGRAVLVHCHAGVSRSVAIITAFLMKTDQLPFEKAYEKLQILKPEAKMNEGFEWQLKLYQAMGYEVDTSSAIYKQYRLQKVTEKYPELQNLPQELFAVDPTTVSQGLKDEVLYKCRKCRRSLFRSSSILDHREGSGPIAFAHKRMTPSSMLTTGRQAQCTSYFIEPVQWMESALLGVMDGQLLCPKCSAKLGSFNWYGEQCSCGRWITPAFQIHKNRVDEMKILPVLGSQTGKI

SEQ ID NO: 147 - protein, 188 aa, Artificial Sequence [sequence omitted]

SEQ ID NO: 148 - protein, 198 aa, Artificial Sequence
  Amino acid sequence of PTP active domain p44
MDSLQKQDLRRPKIHGAVQASPYQPPTLASLQRLLWVRQAATLNHIDEVWPSLFLGDAYAARDKSKLIQLGITHVVNAAAGKFQVDTGAKFYRGMSLEYYGIEADDNPFFDLSVYFLPVARYIRAALSVPQGRVLVHCAMGVSRSATLVLAFLMIYENMTLVEAIQTVQAHRNICPNSGFLRQLQVLDNRLGRETGRF

SEQ ID NO: 149 - protein, 157 aa, Artificial Sequence
  Amino acid sequence of PTP active domain p21
MGNGMTKVLPGLYLGNFIDAKDLDQLGRNKITHIISIHESPQPLLQDITYLRIPVADTPEVPIKKHFKECINFIHCCRLNGGNCLVHCFAGISRSTTIVTAYVMTVTGLGWRDVLEAIKATRPIANPNPGFRQQLEEFGWASSQKLRRQLEERFGES

SEQ ID NO: 150 - protein, 188 aa, Artificial Sequence
  Amino acid sequence of PTP active domain pk35
MTAPSCAFPVQFRQPSVSGLSQITKSLYISNGVAANNKLMLSSNQITMVINVSVEVVNTLYEDIQYMQVPVADSPNSRLCDFFDPIADHIHSVEMKQGRTLLHCAAGVSRSAALCLAYLMKYHAMSLLDAHTWTKSCRPIIRPNSGFWEQLIHYEFQLFGKNTVHMVSSPVGMIPDIYEKEVRLMIPL

SEQ ID NO: 151 - protein, 217 aa, Artificial Sequence
  Amino acid sequence of PTP active domain NE1
MYSLNQEIKAFSRNNLRKQCTRVTTLTGKKIIETWKDARIHVVEEVEPSSGGGCGYVQDLSSDLQVGVIKPWLLLGSQDAAHDLDTLKKNKVTHILNVAYGVENAFLSDFTYKSISILDLPETNILSYFPECFEFIEEAKRKDGVVLVHCNAGVSRAAAIVIGFLMNSEQTSFTSAFSLVKNARPSICPNSGFMEQLRTYQEGKESNKCDRIQENSS

SEQ ID NO: 152 - protein, 190 aa, Artificial Sequence
  Amino acid sequence of PTP active domain p19
MTASASSFSSSQGVQQPSIYSFSQITRSLFLSNGVAANDKLLLSSNRITAIVNASVEVVNVFFEGIQYIKVPVTDARDSRLYDFFDPIADLIHTIDMRQGRTLLHCMAGVSRSASLCLAYLMKYHSMSLLDAHTWTKSRRPIIRPNNGFWEQLINYEFKLFNNNTVRMINSPVGNIPDIYEKDLRMMISM

SEQ ID NO: 153 - protein, 184 aa, Artificial Sequence
  Amino acid sequence of PTP active domain pk18
MGNGMNKILPGLYIGNFKDARDAGQLSRNKVTHILSVHDXXXXXXXGVKYLCIPAADSPSQNLTRHFKESIKFIHECRLRGESCLVHCLAGVSRSVTLVIAYIMTVTDFGWEDALHTVRAGRSCANPNVGFQRQLQEFEKHEVHQYRQWLKEEYGESPLQDAEEAKNILAAPGIMKFWAFLRRL

SEQ ID NO: 154 - protein, 171 aa, Artificial Sequence
  Amino acid sequence of PTP active domain p12
GRAHRDWYHRIDPTVLLGALPLRSLTRQLVQDENVRGVITMNEEYETRFLCNSSQEWKRLGVEQLRLSTVDMTGIPTLDNLQKGVQFALKYQSLGQCVYVHCKAGRSRSATMVAAYLIQVHKWSPEEAVRAIAKIRSYIHIRPGQLDVLKEFHKQITARATKDGTFVISKT

SEQ ID NO: 155 - protein, 177 aa, Artificial Sequence [sequence omitted]

SEQ ID NO: 156 - protein, 150 aa, Artificial Sequence
  Amino acid sequence of PTP active domain p16
MGVQPPNFSWVLPGRLAGLALPRLPAHYQFLLDLGVRHLVSLTERGPPHSDSCPGLTLHRLRIPDFCPPAPDQIDRFVQIVDEANARGEAVGVHCALGFGRTGTMLACYLVKERGLAAGDAIAEIRRLRPGSIETYEQEKAVFQFYQRTK

SEQ ID NO: 157 - protein, 210 aa, Artificial Sequence
  Amino acid sequence of PTP active domain T16
MAHNKIPPRWLNCPRRGQPVAGRFLPLKTMLGPRYDSQVAEENRFHPSMLSNYLKSLKVKMGLLVDLTNTSRFYDRNDIEKEGIKYIKLQCKGHGECPTTENTETFIRLCERFNERNPPELIGVHCTHGFNRTGFLICAFLVEKMDWSIEAAVATFAQARPPGIYKGDYLKELFRRYGDIEEAPPPPLLPDWCFEDDEDEDEDEDGKKES

SEQ ID NO: 158 - protein, 152 aa, Artificial Sequence
  Amino acid sequence of PTP active domain p18
MLLILGQMDKPSLIFDHLYLGSEWNASNLEELQGSGVDYILNVTREIDNFFPGLFAYHNIRVYDEETTDLLAHWNEAYHFINKAKRNHSKCLVHCKMGVSRSASTVIAYAMKEFGWPLEKAYNYVKQKRSITRPNAGFMRQLSEYEGILDAS

SEQ ID NO: 159 - protein, 145 aa, Artificial Sequence
  Amino acid sequence of PTP active domain NE3
MDSPTQIFEHVFLGSEWNASNLEDLQNRGVRYILNVTREIDNFFPGVFEYHNIRVYDEEATDLLAYWNDTYKFISKAKKHGSKCLVHCKMGVSRSASTVIAYAMKEYGWNLDRAYDYVKERRTVTKPNPSFMRQLEEYQGILLAR

SEQ ID NO: 160 - protein, 160 aa, Artificial Sequence
  Amino acid sequence of PTP active domain pk3
MNRPAPVEVTYKNMRFLITHNPTNATLNKFIEELKKYGVTTIVRVCEATYDTTLVEKEGIHVLDWPFDDGAPPSNQIVDDWLSLVKIKFREEPGCCIAVHCVAGLGRAPVLVALALIEGGMKYEDAVQFIRQKRRGAFNSKQLLYLEKYRPKMRLRFKDS

SEQ ID NO: 161 - protein, 154 aa, Artificial Sequence
  Amino acid sequence of PTP active domain p49
MRFLITHNPTNATLNKFTEELKKYGVTTLVRVCDATYDKAPVEKEGIHVLDWPFDDGAPPPNQIVDDWLNLLKTKFREEPGCCVAVHCVAGLGRAPVLVALALIECGMKYEDAVQFIRQKRRGAFNSKQLLYLEKYRPKMRLRFRDTNGHCCVQ

SEQ ID NO: 162 - protein, 158 aa, Artificial Sequence
  Amino acid sequence of PTP active domain p26
MNRPAPVEVSYKHMRFLITHNPTNATLSTFIEDLKKYGATTVVRVCEVTYDKTPLEKDGITVVDWPFDDGAPPPGKVVEDWLSLVKAKFCEAPGSCVAVHCVAGLGRAPVLVALALIESGMKYEDAIQFIRQKRRGAINSKQLTYLEKYRPKQRLRFK

SEQ ID NO: 163 - protein, 355 aa, Artificial Sequence
  Amino acid sequence of PTP active domain T29
QDPRRRDPQDDVYLDITDRLCFAILYSRPKSASNVHYFSIDNELEYENFYADFGPLNLAMVYRYCCKINKKLKSITMLRKKIVHFTGSDQRKQANAAFLVGCYMVIYLGRTPEEAYRILIFGETSYIPFRDAAYGSCNFYITLLDCFHAVKKAMQYGFLNFNSFNLDEYEHYEKAENGDLNWIIPDRFIAFCGPHSRARLESGYHQHSPETYIQYFKNHNVTTIIRLNKRMYDAKRFTDAGFDHHDLFFADGSTPTDAIVKEFLDICENAEGAIAVHCKAGLGRTGTLIACYIMKHYRMTAAETIAWVRICRPGSVIGPQQQFLVMKQTNLWLEGDYFRQKLKGQENGQHRAAFS

SEQ ID NO: 164 - protein, 158 aa, Artificial Sequence
  Amino acid sequence of PTP active domain T46
MAEQATKSVLFVCLGNICRSPIAEAVFRKLVTDQNISENWVIDSGAVSDWNVGRSPDPRAVSCLRNHGIHTAHKARQITKEDFATFDYILCMDESNLRDLNRKSNQVKTCKAKIELLGSYDPQKQLIIEDPYYGNDSDFETVYQQCVRCCRAFLEKAH

SEQ ID NO: 165 - protein, 188 aa, Artificial Sequence
  Amino acid sequence of PTP active domain pk1
LIGDFSKGYLFHTVAGKHQDLKYISPEIMASVLNGKFANLIKEFVIIDCRYPYEYEGGHIKGAVNLHMEEEVEDFLLKKPIVPTDGKRVIVVFHCEFSSERGPRMCRYVRERDRLGNEYPKLHYPELYVLKGGYKEFFMKCQSYCEPPSYRPMHHEDFKEDLKKFRTKSRTWAGEKSKREMYSRLKKL

SEQ ID NO: 166 - protein, 189 aa, Artificial Sequence
  Amino acid sequence of PTP active domain T47
LIGDYSKAFLLQTVDGKHQDLKYISPETMVALLTGKFSNIVDKFVIVDCRYPYEYEGGHIKTAVNLPLERDAESFLLKSPIAPCSLDKRVILIFHCEFSSERGPRMCRFIRERDRAVNDYPSLYYPEMYILKGGYKEFFPQHPNFCEPQDYRPMNHEAFKDELKTFRLKTRSWAGERSRRELCSRLQDQ

SEQ ID NO: 167 - protein, 194 aa, Artificial Sequence
  Amino acid sequence of PTP active domain T45
GHLIGDFSKVCALPTVSGKHQDLKYVNPETVAALLSGKFQGLIEKFYVIDCRYPYEYLGGHIQGALNLYSQEELFNFFLKKPIVPLDTQKRIIIVFHCEFSSERGPRMCRCLREEDRSLNQYPALYYPELYILKGGYRDFFPEYMELCEPQSYCPMHHQDHKTELLRCRSQSKVQEGERQLREQIALLVKDMSP

SEQ ID NO: 168 - protein, 271 aa, Artificial Sequence
  Amino acid sequence of PTP active domain Eya2
ERVFVWDLDETIIIFHSLLTGTFASRYGKDTTTSVRIGLMMEEMIFNLADTHLFFNDLEDCDQIHVDDVSSDDNGQDLSTYNFSADGFHSSAPAANLCLGSGVHGGVDWMRKLAFRYRRVKEMYNTYKNNVGGLIGTPKRETWLQLRAELEALTDLWLTHSLKALNLINSRPNCVNVLVTTTQLIPALAKVLLYGLGSVFPIENIYSATKTGKESCFERIMQRFGRKAVYVVIGDGVEEEQGAKKHNMPFWRISCHADLEALRHALELEYL

SEQ ID NO: 169 - protein, 230 aa, Homo sapiens
KQSTPMGLSLPLSTSVPDSAESGCSSCSTPLYDQGGPVEILPFLYLGSAYHASRKDMLDALGITALINVSANCPNHFEGHYQYKSIPVEDNHKADISSWFNEAIDFIDSIKNAGGRVFVHCQAGISRSATICLAYLMRTNRVKLDEAFEFVKQRRSIISPNFSFMGQLLQFESQVLAPHCSAEAGSPAMAVLDRGTSTTTVFNFPVSIPVHSTNSALSYLQSPITTSPSC

SEQ ID NO: 170 - protein, 235 aa, Homo sapiens
KTKALAAIPPPVPPSATEPLDLGCSSCGTPLHDQGGPVEILPFLYLGSAYHAARRDMLDALGITALLNVSSDCPNHFEGHYQYKCIPVEDNHKADISSWFMEAIEYIDAVKDCRGRVLVHCQAGISRSATICLAYLMMKKRVRLEEAFEFVKQRRSIISPNFSFMGQLLQFESQVLATSCAAEAASPSGPLRERGKTPATPTSQFVFSFPVSVGVHSAPSSLPYLHSPITTSPSC

SEQ ID NO: 171 - protein, 243 aa, Homo sapiens
DVKPISQEKIESERALISQCGKPVVNVSYRPAYDQGGPVEILPFLYLGSAYHASKCEFLANLHITALLNVSRRTSEACATHLHYKWIPVEDSHTADISSHFQEAIDFIDCVREKGGKVLVHCEAGISRSPTICMAYLMKTKQFRLKEAFDYIKQRRSMVSPNFGFMGQLLQYESEILPSTPNPQPPSCQGEAAGSSLIGHLQTLSPDMQGAYCTFPASVLAPVPTHSTVSELSRSPVATATSC

SEQ ID NO: 172 - protein, 170 aa, Homo sapiens
EAPAPALPPTGDKTSRSDSRAPVYDQGGPVEILPYLFLGSCSHSSDLQGLQACGITAVLNVSASCPNHFEGLFRYKSIPVEDNQMVEISAWFQEAIGFIDWVKNSGGRVLVHCQAGISRSATICLAYLMQSRRVRLDEAFDFVKQRRGVISPNFSFMGQLLQFETQVLCH

SEQ ID NO: 173 - protein, 233 aa, Homo sapiens
TNLDGSCSSSSPPLPVLGLGGLRISSDSSSDIESDLDRDPNSATDSDGSPLSNSQPSFPVEILPFLYLGCAKDSTNLDVLEEFGIKYILNVTPNLPNLFENAGEFKYKQIPISDHWSQNLSQFFPEAISFIDEARGKNCGVLVHCLAGISRSVTVTVAYLMQKLNLSMNDAYDIVKMKKSNISPNFNFMGQLLDFERTLGLSSPCDNRVPAQQLYFTTPSNQNVYQVDSLQST

SEQ ID NO: 174 - protein, 232 aa, Homo sapiens
TNVDSSSSPSSSPPTSVLGLGGLRISSDCSDGESDRELPSSATESDGSPVPSSQPAFPVQILPYLYLGCAKDSTNLDVLGKYGIKYILNVTPNLPNAFEHGGEFTYKQIPISDHWSQNLSQFFPEAISFIDEARSKKCGVLVHCLAGISRSVTVTVAYLMQKMNLSLNDAYDFVKRKKSNISPNFNFMGQLLDFERTLGLSSPCDNHASSEQLYFSTPTNHNLFPLNTLEST

SEQ ID NO: 175 - protein, 245 aa, Homo sapiens
TSLAGRAGSSMAPVPGPVPVVGLGSLCLGSDCSDAESEADRDSMSCGLDSEGATPPPVGLRASFPVQILPNLYLGSARDSANLESLAKLGIRYILNVTPNLPNFFEKNGDFHYKQIPISDHWSQNLSRFFPEAIEFIDEALSQNCGVLVHCLAGVSRSVTVTVAYLMQKLHLSLNDAYDLVKRKKSNISPNFNFMGQLLDFERSLRLEERHSQEQGSGGQASAASNPPSFFTTPTSDGAFELAPT

SEQ ID NO: 176 - protein, 252 aa, Homo sapiens
GKPAALLPMSLSQPCLPVPSVGLTRILPHLYLGSQKDVLNKDLMTQNGISYVLNASNSCPKPDFICESRFMRVPINDNYCEKLLPWLDKSIEFIDKAKLSSCQVIVHCLAGISRSATIAIAYIMKTMGMSSDDAYRFVKDRRPSISPNFNFLGQLLEYERSLKLLAALQGDPGTPSGTPEPPPSPAAGAPLPRLPPPTSESAATGNAAAREGGLSAGGEPPAPPTPPATSALQQGLRGLHLSSDRLQDTNRL

SEQ ID NO: 177 - protein, 254 aa, Homo sapiens
GKSTLVPTCISQPCLPVANIGPTRILPNLYLGCQRDVLNKELMQQNGIGYVLNASNTCPKPDFIPESHFLRVPVNDSFCEKILPWLDKSVDFIEKAKASNGCVLVHCLAGISRSATIAIAYIMKRMDMSLDEAYRFVKEKRPTISPNFNFLGQLLDYEKKIKNQTGASGPKSKLKLLHLEKPNEPVPAVSEGGQKSETPLSPPCADSATSEAAGQRPVHPASVPSVPSVQPSLLEDSPLVQALSGLHLSADRLE

SEQ ID NO: 178 - protein, 197 aa, Homo sapiens
NSLQLQECREVGGGASAASSLLPQPIPTTPDIENAELTPILPFLFLGNEQDAQDLDTMQRLNIGYVINVTTHLPLYHYEKGLFNYKRLPATDSNKQNLRQYFEEAFEFIEEAHQCGKGLLIHCQAGVSRSATIVIAYLMKHTRMTMTDAYKFVKGKRPIISPNLNFMGQLLEFEEDLNNGVTPRILTPKLMGVETVV

SEQ ID NO: 179 - protein, 220 aa, Homo sapiens
QKIIWMPQELDAFQPYPIEIVPGKVFVGNFSQACDPKIQKDLKIKAHVNVSMDTGPFFAGDADKLLHIRIEDSPEAQILPFLRHMCHFIEIHHHLGSVILIFSTQGISRSCAAIIAYLMHSNEQTLQRSWAYVKKCKNNMCPNRGLVSQLLEWEKTILGDSITNIMDPLYVGSQSSFSGSMEIIEVSFFMGQLLEFEEDLNNGVTPRILTPKLMGVETVV